(19) United States
(12) Patent Application Publication
(10) Pub. No.: US 2003/0139885 A1
(43) Pub. Date: Jul. 24, 2003

(54) Methods and devices for proteomics data complexity reduction

(75) Inventors: Ansgar Brock, San Diego, CA (US); David M. Horn, San Diego, CA (US); Eric C. Peters, Carlsbad, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (GB)

(21) Appl. No.: 10/289,462

(22) Filed: Nov. 5, 2002

Related U.S. Application Data
(60) Provisional application No. 60/332,988, filed on Nov. 5, 2001.
(60) Provisional application No. 60/368,342, filed on Mar. 27, 2002.
(60) Provisional application No. 60/385,769, filed on Jun. 3, 2002.
(60) Provisional application No. 60/385,364, filed on Jun. 3, 2002.
(60) Provisional application No. 60/385,835, filed on Jun. 3, 2002.
(60) Provisional application No. 60/386,915, filed on Jun. 5, 2002.
(60) Provisional application No. 60/410,382, filed on Sep. 12, 2002.

(51) Int. Cl. G06F 19/00; G01N 33/48; G01N 33/50

(52) U.S. Cl. 702/19

(57) ABSTRACT

Provided are methods and systems for identification of proteins using high mass accuracy mass spectrometry. Not only do high mass accuracy measurements provide greater confidence in protein identification assignments, but they also enable proteins to be identified with either less sequence coverage or fewer additional tandem MS experiments. In addition, high mass measurement accuracy optionally allows protein identifications to be made on the basis of the mass of a single peptide, providing higher-throughputs in the analysis of mixtures due to the significant decrease in time spent on additional tandem MS experiments. In addition, a concomitant time saving in the cross correlation process of mass spectral data with in silico digested databases would also be achieved.

### CROSS-REFERENCE TO RELATED APPLICATIONS

This application is related to U.S. provisional patent applications U.S. S No. 60/368,342 filed Mar. 27, 2002; U.S. S No. 60/385,769 filed Jun. 3, 2002; and U.S. S No. 60/385,364 filed Jun. 3, 2002. This application is also related to U.S. provisional patent applications U.S. S No. 60/332,988 filed Nov. 5, 2001; U.S. S No. 60/385,835 filed Jun. 3, 2002; and U.S. S No. 60/410,382 filed Sep. 12, 2002, titled “Labeling Reagent and Methods of Use”; and U.S. S No. 60/386,915 filed Jun. 5, 2002 and titled “Sample Preparation Methods for MALDI Mass Spectrometry.” The present application claims priority to, and benefit of, these applications, pursuant to 35 U.S.C. §19(e) and any other applicable statute or rule.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to analysis of protein samples by mass spectrometry. More particularly, the present invention relates to methods for reducing data complexity in proteomic samples, and protein identification using isotopic labeling and/or high mass accuracy mass spectrometric techniques.

### BACKGROUND OF THE INVENTION

A number of sophisticated approaches have been developed to study the structure and function of genes, including the whole-scale sequencing of entire organisms, global transcriptional profiling, and forward genetic studies. However, these techniques are ultimately limited by the fact that they only assess intermediates on the way to the protein products that ultimately regulate biological processes. Processes such as RNA processing, proteolytic activation, and hundreds of possible post-translational modifications (PTMs) can result in the production of numerous proteins of unique structure and function from a limited number of genes. Additionally, biological activity often results from the assembly of numerous proteins into an active complex, the nature and composition of which can only be explored at the protein level.

Proteomics is the study of the “proteome,” the protein complement expressed by a genome at a given point in time. Proteomic studies should be able to answer many questions about cellular processes and diseases that can't be answered by genomic methods alone. However, such studies are more difficult to perform than their genomic counterparts, and any general analysis platform must possess high sensitivity, be tolerant of a wide range of experimental and analytical conditions, and be able to process and display massive amounts of information. In addition, these analysis systems must also be able to perform extremely high-throughput measurements, since, unlike the relatively fixed nature of the genome, the expression and interactions of proteins are in a constant state of flux, varying over time, tissue type, and in response to environmental changes.

Historically, two-dimensional gel electrophoresis (2DE) has been the dominant technique for assessing large-scale changes in protein expression patterns. The development and emergence of biological mass spectrometry (MS) in the early 1990's greatly increased the amount of information obtained using two-dimensional gel electrophoresis, enabling the identification of thousands of encoded proteins by peptide mapping and/or tandem MS experiments (for general reviews see Karas and Hillenkamp (1988) “Laser desorption ionization of proteins with molecular masses exceeding 10,000 daltons” Anal. Chem. 60:2299-2301; Fenn et al. (1989) “Electrospray Ionization for Mass Spectrometry of Large Biomolecules” Science 246:64-71; and Patterson and Aebersold (1995) “Mass spectrometric approaches for the identification of gel-separated protein” Electrophoresis 16:1791-1814). Although powerful, these techniques remain laborious, and possesses several widely recognized limitations, including the difficulty of comparing results between laboratories, operational difficulty in handling certain classes of proteins, and potential unwanted chemical modifications. An additional shortcoming of the classic 2DE technique is its inability to accommodate the extreme range of protein expression levels inherent in complex living organisms due to sample loading restrictions imposed by the gel-based separation technology employed. This limitation is of particular concern since many proteins of interest (e.g., regulatory proteins) are often expressed at low copy numbers per cell. Extensive protein prefractionation schemes based on differing solubility, isoelectric points, or subcellular locations have been proposed to address the problem of analyzing low abundance proteins. However, questions remain as to whether the integrity of the original protein mixture can be maintained. In addition, any of these approaches greatly increase the number of (relatively slow) 2DE experiments that need to be performed, reducing the feasibility of a proteomics approach.

Multi-dimensional chromatography combined with MS and/or tandem MS methods has been explored as an alternative method to explore the proteome (see, for example, Yates (2000) “Mass spectrometry: from genomics to proteomics” Trends. Genet. 16:5-8; Aebersold and Goodlett (2001) “Mass spectrometry in proteomics” Chem. Rev. 101:269-95). Samples are partially purified and separated by one or more liquid chromatographic techniques, the fractions from which are then analyzed and identified by separating gaseous ions of the substances according to their mass-to-charge ratio. The chromatographic separations serve to disperse the complexity of the initial sample, and can be performed at both the peptide as well as at the protein level (although protein identification is typically performed using peptides). The information gleaned from MS experiments of an analyte mixture can be further refined based on the presence of particular amino acids or specific post-translational modifications (see, for example, Wang and Regnier (2001) “Proteomics based on selecting and quantifying cysteine containing peptides by covalent chromatography” J. Chromatogr. A 924:345-57; Ji et al. (2000) “Strategy for quantitative and qualitative analysis in proteomics based on signature peptides” J. Chromatogr. B 745:197-210; Gygi et al. (1999) Nat. Biotechnol. 17:994-9.; and Cao and Stults (1999) “Phosphopeptide analysis by on-line immobilized metal-ion affinity chromatography-capillary electrophoresis-electrospray ionization mass spectrometry” J. Chromatography A 853:225-235). Similarly, MS techniques have been developed for quantitatively assessing a differential display of proteins or PTMs (see Martin et al. (2000) “Sub-femtomole MS and MS/MS peptide sequence analysis using nano-HPLC micro-ESI Fourier transform ion cyclotron resonance mass spectrometry” Anal. Chem. 72:4266-74; Blume-Jensen and Hunter (2001) “Oncogenic kinase signaling” Nature 411:355-65; Goshe et al. (2001) “Phosphoprotein isotope-coded affinity tag approach for isolating and quantitating phosphopeptides in proteome-wide analyses” Anal. Chem. 73:2578-86; and Oda et al. (2001) “Enrichment analysis of phosphorylated proteins as a tool for probing the phosphoproteome” Nat. Biotechnol. 19:379-82).

Electrospray ionization (ESI) methods are most commonly employed, due in part to the simplicity of their implementation. However, parameters for coupling LC and ESI mass spectrometry impose several undesirable limitations, making this technique less suitable for proteomics experiments. Specifically, the separation system and mass spectrometer employed are coupled directly in real time, making the construction of parallel analysis systems difficult (or at least extremely costly), and often preventing the mass spectrometer from continually collecting useful data due to the equilibration and washing periods typical of separation techniques. More importantly, current instrument control and data analysis software is not nearly fast enough to allow real time data-dependent processing during the course of a chromatographic separation except when employing simple selection criteria such as peak intensity. This necessitates that upon the completion of a separation and subsequent analysis of the resulting data, the same sample must be rerun to focus on those species that exhibited the desired selection criteria (see Pieper et al. (1999) “Biochemical identification of a mutated human melanoma antigen recognized by CD4+ T cells” J. Exp. Med. 189:757-66). Additionally, monitoring the levels of several particular species over time requires the active engagement of the mass spectrometer over the whole course of the chromatographic run, even though the species of interest themselves elute only in specific narrow time windows throughout the gradient profile. Ultimately, these and other limitations result in dramatic reductions in overall platform throughput.

### SUMMARY OF THE INVENTION

The complexity and magnitude of data generated during MS proteomic studies provokes the need for powerful analytical platforms for managing, assessing and analyzing the volume of data generated. The present invention provides novel methods and integrated systems that address this need in the art, in part through the use of high mass accuracy measurements as can be obtained by FT-ICR MS, in combination with data reduction processes.

In a first aspect, the present invention provides methods for reducing a number of peaks to be further analyzed (e.g. unidentified peaks) in a mass spectrum or MS data set generated for a sample. The methods include the steps of: a) generating a first amino acid sequence database comprising an amino acid sequence of at least one protein known (or assumed) to be present in the sample; b) calculating a first list of theoretical masses for a first set of in silico peptides generated from one or more of the amino acid sequences in the first database; and c) correlating the first list of theoretical masses with positions of the unidentified MS peaks and identifying one or more MS peaks that correspond to masses for the in silico peptides, thereby reducing the number of peaks to be further analyzed in the mass spectrum. If the sample proteins were treated with a proteolytic agent prior to generating the mass spectrum, the in silico peptides are generated using the same proteolytic cleavage parameters. In order to perform the comparison, the unidentified MS peaks are preferably obtained using a mass spectrometer that provides a high mass accuracy, for example, a mass accuracy of 5 ppm or better, or more preferably of 1 ppm or better. The list of experimental mass peaks can be provided by a single MS spectrum or by a set of MS spectra (e.g., a compiled data set).

Optionally, all members of the first database of amino acid sequences are derived from proteins known to be present in the sample (i.e., the database consists of amino acid sequences from one or more proteins known to be present in the sample). The first sequence database can be introduced from experimental data previously used to assign a portion of the proteins present in the sample, such as protein sequencing data, nucleic acid sequencing data, tandem MS data, 2DE-MS data, and the like. In one embodiment, generating the first database comprises i) selecting an unidentified MS peak and performing tandem mass spectrometry, thereby identifying a corresponding peptide sequence; and ii) determining a parent protein sequence comprising the identified corresponding peptide sequence. In silico peptides representing additional portions of the parent protein are generated, from which the first list of theoretical masses is then calculated. By correlating the first list of theoretical masses with positions of the unidentified MS peaks, additional experimental peaks representing these additional peptides of the identified protein are resolved. These additional MS peaks can be removed from the list of unidentified MS peaks (since they are fragments of the previously identified protein), thereby reducing the number of unidentified peaks in the mass spectrum (and the complexity of the spectrum).

Alternatively, the database of proteins from which the theoretical peptide masses are calculated can be generated by a more brute force approach. In this embodiment, generating the database includes i) providing a mass peak list comprising the positions of the unidentified MS peaks of the sample, wherein the MS peaks represent a plurality of proteolytic peptides generated by action of a proteolytic agent upon member proteins in the sample; ii) providing a second list of theoretical masses for a plurality of in silico proteolytic peptides generated from a second database of protein sequences by the in silico action of the proteolytic agent (e.g., using the same cleavage parameters) upon member sequences in the second database; and iii) comparing the second list with the mass peak list, thereby assigning corresponding MS peaks and identifying member proteins of the sample for inclusion in the first database. The database generated thus, a veritable universe of peptide fragments, is then compared to the MS data for the sample. In this manner, corresponding MS peaks are assigned and additional member proteins of the sample are identified for inclusion in the first database. This approach can be used to “weed out” the MS peaks representing more common peptide fragments (as would be generated by using a broadly inclusive database of protein sequences), thus significantly reducing the complexity of the remaining spectrum of unidentified peaks.

In some embodiments, the plurality of in silico peptides used to generate the list of theoretical molecular masses employed in the methods is limited in scope by one or more constraints. The member peptides optionally can be limited to a selected size range (for example, ranging from 1000 Da to 4000 Da or 6000 Da). The peptides can be limited in composition (e.g., having a particular amino acid constituent or sequence motif). Theoretical mass calculations can be performed only on fragments as generated in silico by a specific proteolysis reaction, and can optionally take into account “missed” cleavage sites. For derivatized peptides (as described below), the mass calculation should also take into account the presence of the derivatizing moiety.

In a further embodiment of the methods of the present invention, the list of theoretical molecular masses is limited to include only unique masses arising for distinct peptide fragments (i.e., each mass in the list of theoretical masses corresponds to one and only one unique peptide sequence). In this embodiment, correlation of an experimental peak with a unique mass from the list of theoretical masses provides an identification of the peptide (and the corresponding parent protein).

The data complexity reduction methods of the present invention can optionally be performed in an iterative manner, to further assign the unidentified MS peaks based upon information gleaned from the previous round of analysis. In this embodiment, after identification of one or more parent protein sequences (for example, by correlating an MS peak with a unique theoretical mass), the first database of identified proteins is regenerated to include the newly identified parent protein sequences (e.g., additional member proteins). Additional in silico peptide fragments are generated from the information in the updated first database, and the corresponding (unique and/or non-unique) theoretical masses are again compared to the list of mass peaks for the sample, to further reduce the number of unidentified MS peaks and to possibly correlate unassigned MS peaks to further additional parent proteins. The steps of regenerating the list of parent proteins, calculating theoretical masses for component peptides, and correlating the list to the remaining unidentified MS peaks is optionally repeated until no additional member proteins are identified.

Optionally, the member proteins in the sample (or proteolytically-cleaved fragments thereof) can be isotopically labeled prior to generating the mass list, to further assist in the assignment of the MS peaks. In these embodiments, the sample is contacted with a first derivatizing agent having at least two isotopic forms to label the member proteins at one or more selected amino acids or selected functionality groups. Contacting the sample with the derivatizing agent can be performed before or after preparation and/or optional fractionation of the sample. In one embodiment of the present invention, proteins in the sample are labeled by performing a chemical reaction that alters the molecular mass of the protein or proteolytic peptide. In an alternate embodiment, cells are grown in the presence of the isotopically-labeled derivatization agent (e.g., an isotopically-labeled amino acid or amino acid precursor), thereby labeling the proteins in situ. Both approaches are considered embodiments of contacting the sample with the first derivatizing agent. Preferably, MS data on the isotopically-labeled sample is collected using a mass spectrometer that provides a mass accuracy of 5 ppm or better, such as a Fourier-transform ion cyclotron resonance mass spectrometer.

In addition, the methods of the present invention can be used to assign MS peaks from proteolytically-cleaved peptides having mass-altering modifications besides (or in addition to) isotopic labeling, such as peptide fragments generated from post-translationally modified proteins. In this embodiment, calculating the first list of theoretical masses (for the proteins identified thus far) involves generating theoretical masses for peptides assumed to contain one or more occurrences of a selected peptide modification. The peptide modification can be a “natural” (e.g., cell-generated) modification (such as a glycosylation, myristoylation, phosphorylation, etc.) or other modification (e.g., addition/substitution involving a standard or non-standard amino acid, isotope-label incorporation, etc.) performed generated during or after peptide synthesis. Alternatively, the modification can be a chemical or synthetic modification generated independent of peptide synthesis (e.g., such as iodination, affinity labeling, chemical labeling, and the like).

The present invention also provides methods for identifying members of a plurality of proteins in a sample. The methods include the steps of: a) contacting a sample comprising a plurality of proteins with at least a first proteolytic agent that cleaves member proteins at defined cleavage sites to form proteolytic peptides; b) contacting the sample with a first derivatizing agent comprising at least two isotopic forms, wherein the first derivatizing agent specifically labels a selected amino acid (or a functional moiety of an amino acid) when the selected amino acid (or functional moiety) is present in a protein in the sample, thereby isotopically labeling one or more members of the plurality of proteins or proteolytic peptides; c) fractionating the sample and depositing a plurality of fractions of an eluent onto a solid support suitable for LDI; d) performing LDI-FT ICR mass spectrometry on the isotopically-labeled peptides in one or more of the fractions and determining masses of at least one pair of peaks of interest using a mass spectrometer that provides a mass accuracy of 5 ppm or better; e) calculating a list of theoretical molecular masses for a plurality of in silico derivatized proteolytic peptides, wherein the member proteolytic peptides i) are derived from the amino acid sequences in a protein sequence database by predicted action of the proteolytic reagent upon members of the database; ii) encompass peptides having up to three missed proteolytic cleavage sites; iii) range in size between 1000 Da and 6000 Da; and iv) comprise one or more derivatized amino acids; and f) correlating the list of theoretical molecular masses to the mass peak list of experimental mass peaks, wherein a match between an experimental mass peak of a sample proteolytic peptide and a theoretical molecular mass for an in silico proteolytic peptide is indicative of the presence in the sample of the protein from which the in silico proteolytic peptide is derived, thereby assigning MS peaks in the mass peak list and identifying the members of the plurality of proteins.

As noted above, the assignments determined in a first round of protein identification can be used to reduce the complexity of the MS data set and facilitate further protein identification. In a further embodiment of the protein identification method, the method includes the steps of: i) removing the assigned MS peaks from the mass peak list; ii) incorporating the identified members of the plurality of proteins into a database of identified proteins; and iii) repeating the calculation and correlating steps using in silico derivatized proteolytic peptides generated from the database of identified proteins, thereby assigning additional MS peaks in the mass peak list and identifying additional members of the plurality of proteins. By determining which MS peaks in the mass peak list represent previously assigned proteins and the removing redundant peaks from the list of unassigned peaks, the resulting mass peak list is reduced in complexity, allowing for MS peak assignment efforts to be focussed primarily on any additional unidentified proteins.

In yet a further embodiment, the protein identification method includes the steps of a) providing one or more additional databases of proteolytic peptide sequences, wherein the member proteolytic peptides i) are derived in silico by predicted action of one or more additional proteolytic reagents upon members sequences in the second database of protein sequences; ii) encompass peptide sequences having up to three missed enzymatic cleavage sites; iii) range in size between 1000 Da and 4000 Da; and iv) comprise one or more derivatized amino acids; and b) repeating the generating and correlating step using the one or more additional databases, thereby identifying additional members of the plurality of proteins.

In a further aspect, the present invention provides additional methods for identifying members of a plurality of proteins. The methods are particularly useful for samples having large numbers of member proteins (e.g., from 50 to 25,000 member proteins). The method employs a set of unique theoretical masses selected from calculated theoretical masses for a plurality of in silico peptides (as described previously); a match between an unidentified experimental MS peak and a unique theoretical molecular mass for an particular in silico proteolytic peptide indicates that the parent protein from which the in silico proteolytic peptide is “derived” is present in the sample, thereby identifying a protein constituent of the sample.

In the simplest embodiment, the protein identification methods include the steps of a) providing a sample that comprises a plurality of proteolytic polypeptides; b) ionizing member polypeptides by LDI and obtaining a mass of at least a first polypeptide using a mass spectrometer that provides a mass accuracy of 5 ppm or better; and c) comparing the mass of the first polypeptide to members of a database of theoretical molecular masses for a plurality of in silico proteolytic peptides, wherein each member in silico peptide has a unique theoretical mass, and wherein a match between the mass obtained for the first polypeptide and the unique theoretical mass for an in silico proteolytic peptide indicates that a parent protein comprising the in silico polypeptide is present in the sample, thereby identifying a first protein in the sample. Optionally, the comparing step is repeated for additional MS peaks in the experimental data set, thereby identifying additional proteins in the sample.

As an additional embodiment, the method includes the steps of a) contacting the plurality of proteins in the sample with a first derivatizing agent, wherein the first derivatization agent comprises at least two isotopic forms and specifically labels a selected amino acid (or a specific functional group) when the selected amino acid is present in a sample protein. The sample is optionally fractionated; in one embodiment, the fractionating step further includes depositing a plurality of fractions of an eluent onto a solid support suitable for laser desorption/ionization (LDI). The member polypeptides in the fractions are ionizing (by ESI, MALDI, or an alternative ionization technique) and a mass is obtained for at least a first polypeptide. Preferably the process is performed using a mass spectrometer that provides a mass accuracy of 5 ppm or better. The mass obtained for a first polypeptide is compared to members of a database of theoretical molecular masses for a plurality of in silico proteolytic peptides that are derived from amino acid sequences for a plurality of proteins. A match between the mass obtained for the polypeptide and the theoretical molecular mass for an in silico proteolytic peptide is indicative of the presence in the sample of the protein from which the in silico proteolytic peptide is derived, thereby identifying a first protein in the sample.

Optionally the comparing step can be repeated for one or more masses obtained for additional polypeptides, thereby identifying additional proteins in the sample. For example, the methods optionally include the steps of e) calculating theoretical molecular masses for one or more additional in silico peptides derived from the protein identified in the comparison of the mass obtained for the first sample peptide to the theoretical molecular masses; and f) subjecting at least a second peptide to mass spectrometry, and disregarding mass spectral data for the second peptide if the mass spectral data for the this peptide matches (e.g., is within 5 ppm of) that which would be obtained for one or more of the additional in silico peptides from the previously identified protein. Thus, data which matches an already-identified protein sequence can be removed from the data set, thereby reducing the population of mass peaks yet to be identified and thereby the overall complexity of the sample. Other parameters can also be used to determine whether spectral data for an additional peptide can be disregarded. For example, an expression ratio determined for the second peptide that corresponds to an expression ratio for the first peptide, or a number of derivatized amino acids of the second peptide that corresponds to a number of theoretical derivatized amino acids for the second in silico peptide, can confirm the decision to remove the MS peak from the list of unassigned peaks.

The present invention also provides integrated systems for identifying member proteins in a sample. The system includes a) an ionization source and a mass spectrometer that provides a mass accuracy of 5 ppm or better; b) an interface for receiving mass spectral data from the mass spectrometer, c) a database of theoretical molecular masses of in silico polypeptides, and d) a computer or computer-readable medium in communication with both the interface and the database of theoretical molecular masses. The computer or computer-readable medium includes instructions for determining the mass of the labeled polypeptide from the mass spectral data. The instructions also provide for comparison between the experimentally-determined mass and the database of theoretical molecular masses, taking into account the (optional) proteolytic treatment as well as any changes in mass due to addition of one or more derivatizing agents. Additional system components optionally include, but are not limited to, a liquid chromatography system for fractionating the sample, an automated sample collection system, an eluent collection plate (e.g., a hydrophobic/hydrophilic MALDI plate), a sample source, a source of one or more proteolytic reagents, one or more mixing regions for contacting the sample with one or more proteolytic reagents and/or derivatizing agents, and one or more additional databases of in silico proteolytic peptides generated by various proteolytic agents.

Preferably, the mass spectrometer component of the integrated system is an FT-ICR mass spectrometer. In a preferred embodiment, the mass spectrometer is configured to analyze ions generated from sample fractions co-crystallized with matrix on the optional eluent collection plate. Optionally, software for controlling generation and processing of the mass spectral data by the mass spectrometer is incorporated into the interface component of the system.

The integrated systems of the present invention can also include a number of mechanisms for addressing differences in mass between (unmodified) amino acid sequences as provided by a protein database (or generated from a nucleic acid database), and the modified, derivatized or otherwise mass-altered peptide present in proteomic (i.e., real-world) samples. For example, the system can account for derivatization-based changes in molecular mass by adjusting the theoretical masses by the mass of the number of derivatizing agents potentially associated with the sequence.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “an analyte” includes a combination of two or more analytes; reference to “a calibrant” includes mixtures of calibrant compounds, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

The term “proteolytic agent” as used herein refers to a moiety (enzyme, chemical, etc.) capable of breaking a peptide bond, preferably in a specific position within the amino acid sequence.

The terms “derivatizing agent” or “derivatization agent” are interchangeably used to refer to a reagent (e.g., a chemical compound, a catalyst, an enzyme, a labeled amino acid or amino acid precursor, etc.) capable of generating a mass-altered amino acid in a peptide (e.g., by binding to, replacing, chemically modifying, and/or labeling an amino acid or a functional moiety of the peptide).

The term “isotopic forms” refers to multiple versions of the derivatizing agent which are identical structurally but differ in isotopic content.

The terms “polypeptide,” “peptide” and “protein” are used interchangeably to include a molecular chain of amino acids linked through peptide bonds. As used herein, the terms do not refer to a specific length of the product. Thus, “peptides,” “o ligopeptides,” and “proteins” are included within the definition of polypeptide. Furthermore, protein fragments, analogs, mutated or variant proteins, fusion proteins and the like are included within the meaning of polypeptide, as well as any chemical or post-translational modifications of the polypeptide, for example, glycosylations, acetylations, esterifications, phosphorylations and the like.

The term “mass accuracy” refers to the absolute value of the difference between the measured mass and the actual exact mass, divided by the actual exact mass: e.g.,

$$mass accuracy = \frac{\vert ( Measured Mass - The Actual Exact Mass ) \vert}{The Actual Exact Mass}$$

The term “matches” when used in conjunction with mass spectral data, refers to values which differ by 5 ppm or less of one another. Thus, the phrase “if the mass spectral data for a first peptide matches that of another peptide” would include data which differ by up to (and including) 5 ppm.

The term “unique mass” as used herein refers to a molecular mass that can only arise from (and be assigned to) to a single peptide or protein in a specified database of peptide or protein sequences.

The term “proteome” refer to the protein constituents expressed by a genome, typically represented at a given point in time. A “sub-proteome” is a portion or subset of the proteome, for example, the proteins involved in a selected metabolic pathway, or a set of proteins having a common enzymatic activity.

As used herein, the terms “non-standard amino acid,” “non-natural amino acid” and “a typical amino acid” interchangeably refers to amino acids other than the 20 primary amino acids typically found in proteins.

### BRIEF DESCRIPTION OF THE DRAWINGS

FIG. 1: Flow chart of an “accurate mass” platform for the protein profiling of biological samples.

FIG. 2 provides a 3-dimensional plot of a reverse phase μHPLC MALDI FT-ICR MS analysis of a tryptic digest of the soluble proteins isolated from yeast.

FIG. 3A shows the effect of having specific amino acid information on proteome coverage for yeast and human.

FIG. 3B shows the effect of mass accuracy on proteome coverage for yeast and human.

FIG. 3C shows the effect of various proteases on proteome coverage for yeast and human. Mass accuracy was 1 ppm, and lysines and acidic residues were derivatized.

FIG. 4 shows the effect of derivatization on the number of identifiable peptides per protein in the human proteome at 1 ppm mass accuracy.

FIG. 5 shows the effect of derivatization on the number of identifiable peptides per protein in the yeast proteome at 1 ppm mass accuracy.

FIG. 6 shows the effect of mass accuracy and derivatization strategy on the percentage of all possible tryptic peptides that can be identified in the yeast proteome.

FIG. 7 shows the effect of mass accuracy and derivatization strategy on the percentage of all possible tryptic peptides that can be identified in the human proteome.

FIG. 8 shows the effect of mass accuracy and derivatization strategy on yeast proteome coverage.

FIG. 9 shows the effect of mass accuracy and derivatization strategy on human proteome coverage.

FIG. 10A depicts the percentage of phosphorylated peptides that are uniquely identifiable in a human proteome sample, given 1 ppm mass accuracy and lysine and acidic amino acid specificity information.

FIG. 10B depicts the percentage of myristoylated peptides that are uniquely identifiable in a human proteome sample, given 1 ppm mass accuracy and lysine and acidic amino acid specificity information.

FIG. 11 depicts mass spectra generated for a sample using MALDI TOF (left) and MALDI FT-ICR.

FIG. 12 provides a SORI-CAD spectrum of an unidentified peptide with mass 1752.58 from a tryptic digest of all soluble cytosolic proteins in yeast.

### DETAILED DESCRIPTION

The present invention provides novel methods and systems for spectral data complexity reduction and/or protein identification using mass spectrometry (MS). The approaches described herein have a number of advantages over the conventional approach of repeatedly performing tandem MS experiments on individual components of large populations of protein sequences, such as proteome samples (see, for example T. Ideker et al. “Integrated Genomic and Proteomic Analyses of a Systematically Perturbed Metabolic Network” (2001) Science 292:929-934).

For example, the methods of the present invention dramatically reduce the time and number of experiments required for identification of large populations of proteins. For a sample as complex as a proteome (e.g., having tens or hundreds of thousands of different proteins), the conventional MS approach requires that all species detected be analyzed by tandem MS, in order to prevent missing the presence of a given peptide. While each tandem MS experiment requires only a few seconds per peptide, tens of thousands of such experiments would need to be performed in the analysis of a complete proteome. Due to this requirement to perform exhaustive tandem MS, conventional systems require further fractionation of the sample, in order to present less complex mixtures at any one time to the instrument, and allow the instrument to perform all of the necessary tandem MS measurements. One advantage of the present invention is that large populations of sequences can be analyzed from data generated by a single MS experiment, thereby reducing the time that would have been spent fractionating sample proteins into smaller (more manageable) populations and collecting multiple MS spectra on the resulting fractions.

An additional advantage to the methods of the present invention relates to sample quantity limitations. There are a limited number of tandem MS experiments that can be performed on a given spot on a target plate before the sample is depleted by the laser desorption process. Since protein identification using the methods of the present invention is performed via deconvolution of the MS data, rather than repeated experiments, the sample fractions can be extended further, or used in alternative experiments. Furthermore, tandem MS is typically an order of magnitude less sensitive than MS due to the splitting of the signal of a single peptide into several daughter ions. Thus, protein identification by the methods and systems of the present invention is not only faster, but also at least an order of magnitude more sensitive than those currently employed in the art.

Complexity Reduction Using Accurate Mass for Proteomics (CRAMP)

In a first aspect, the present invention provides methods of reducing the complexity of a complex data set being analyzed using a mass comparison approach. Since a mass spectrum (or set of spectra) generated for a typical proteomics sample typically contain hundreds or possibly thousands of mass spectral peaks, methods for reducing the complexity of the collected data would be highly advantageous. This can be achieved, via the methods of the present invention, by comparison of the experimental MS data to theoretical peak positions. The methods of the present invention do not require a physical simplification of the sample prior to collecting the mass spectral data; thus, data collection optionally can be performed without further fractionation of the plurality of proteins (or alternatively, data from multiple spectra can be tabulated into a master list of MS peak positions and analyzed together).

The methods of reducing a number of unidentified peaks in a mass spectrum for a sample include the steps of a) generating a first amino acid sequence database comprising at least one protein sequence present in the sample; b) calculating a first list of theoretical masses for a first set of in silico proteolytic peptides generated from the first database; and correlating the first list of theoretical masses with positions of the unidentified MS peaks and identifying one or more peaks that correspond to peptides present in the second database, thereby reducing the number of unidentified peaks in the mass spectrum. Preferably, the unidentified MS peaks were collected using a mass spectrometer that provides a mass accuracy of 5 ppm or better (e.g., a high mass accuracy mass spectrometer, such as a FT-ICR mass spectrometer). The methods of the present invention have not been previously attempted in the prior art due in part to practical constraints; several technical aspects of the platform, such as a) efficient coupling of the chromatography and MS systems, b) ionization techniques capable of introducing the biomolecules into the spectrometer, c) effective methods for internal calibration, and d) sufficient mass accuracy and resolution (i.e. better than 5 ppm accuracy) to make this approach useful have only recently become available. (See, for example, U.S. patent application Ser. No. ______ [Attorney Docket No. 36-003010US] and PCT application ______ [Attorney Docket No. 36-003010PC] co-filed herewith.)

Generating the First Sequence Database

In one embodiment of the present invention, the first round of data simplification is based upon comparison to a list of theoretical masses for expected peptides based upon one or more known protein entities in the sample. The known proteins can be ascertained (and the corresponding first sequence database can be initially generated) by any of a number of mechanisms. For example, one or more peptide sequences can be determined via a tandem MS experiment or a 2DE-MS experiment performed on the sample (or a component thereof). Alternatively, the initial sequences can be derived from protein sequencing data or nucleic acid sequencing data. The sequences for the known proteins can even be selected based upon artificial assumptions of the protein content of the sample (i.e., using the hemoglobin sequence for a sample derived from a red blood cell), or motif searches (e.g., glycosylation sites, ligand binding sites, etc.).

As an alternative approach, generating the first database of identified protein sequences can include a) providing a mass peak list generated from the experimental data; b) providing a second list of theoretical masses generated in silico from a second protein sequence database; and c) comparing the second list with the mass peak list. In this embodiment, the comparison of sample peaks to a database of peptide sequence peak positions (e.g., the universe of peptides available) can be used to make the first assignments in the experimental mass spectrum. As noted previously, the data used to generate the experimental list of MS peaks need not come from a single spectrum; optionally, the data can be compiled from multiple MS spectra for the sample (e.g., from multiple fractions of the sample, or multiple spots on a MALDI sample support).

The second list of theoretical masses are derived from a second database of protein sequences, or optionally from a database of corresponding nucleic acid sequences. In some embodiments of the present invention, the second database is a large (e.g., fairly inclusive) public or commercially-available sequence database. Alternatively, the second database of protein sequences can be generated from laboratory sequencing results, published records, private databases, Internet listings, and the like. A second list of theoretical masses representing a plurality of in silico proteolytic peptides is then generated using entries in the second database of protein sequences.

In one embodiment of the present invention, the mass entries in the second in silico-derived list are considered a single pool. Alternatively, the masses can be compared to the protein sequences from which they are derived, and subdivided into two categories: unique masses that can only be due to a single peptide in the database of sequences, and non-unique masses that could represent any of a number of non-identical peptide sequences in the database. In an alternative embodiment of the present invention, only the unique masses are compared to the MS peaks, thereby providing an added assurance that a correlation between experimental and theoretical MS data is truly represented by the identified sequence. For this embodiment, the method for this “unique mass” aspect of data complexity reduction and protein identification includes the steps of a) providing a mass peak list comprising the positions of the unidentified MS peaks of the sample; b) providing a second list of theoretical masses for a plurality of in silico peptide or protein sequences (from a second database), wherein the second list comprises a first set of unique masses representing unique peptide sequences and a second set of masses representing more than one peptide sequence; and c) comparing the first set of unique masses with the mass peak list, wherein a match between an experimental MS peak and a theoretical mass is indicative of the present of the peptide and/or the protein from which it was derived in the sample. Optionally, the MS peaks (and the theoretical masses of the in silico peptides) represent a plurality of proteolytic peptides generated by action of a proteolytic agent upon member proteins in the sample or in silico database.

It should be noted that, in addition to being used to reduce data complexity, correlation of the experimental MS peaks to the theoretical in silico peptide masses also can be used to identify member proteins of the sample (which aspect is described in further detail below). Even if the assignments are made using the non-unique mass data, it is highly unlikely that a sample protein will not eventually be identified during the methods of the present invention, since alternative peptide fragments will also be identified and assigned. However, additional experiments, such as tandem MS, can optionally be performed to confirm any questionable MS peak assignments. The number of experiments necessary for the few questionable situations would not have nearly as dramatic an effect on throughput as compared to performing all of the identifications by tandem MS.

Typically, both the mass list of experimental MS peaks and the second list of theoretical masses represent a plurality of proteolytic peptides generated by action of a proteolytic agent upon member sequences. Optionally, the mass data also reflects additional criteria beyond the presence of a proteolytic cleavage site. For example, the second list of in silico theoretical masses can include masses for polypeptides that were incompletely cleaved due to missed cleavage sites (as happens in the real world). Optionally, the database can include up to one, two, three, or more missed cleavages per peptide sequence. As another option, the database of sequences can be limited in size, for example, to include only peptides that fall within a selected size range. In addition, the database can be selected to include only peptides having a selected amino acid. Furthermore, any combination of these (or other) criteria can be applied to the databases employed in the present invention.

In a further embodiment, the methods for reducing data complexity as provided herein can be performed in an iterative manner. After correlating some of the experimental mass peaks with their corresponding peptide in the second database, the newly-identified proteins from the second database are added to the first database of identified proteins, thereby regenerating the first database. Additional proteolytic peptide masses are determined based upon the new members of the first database, and the calculating and correlating steps are repeated to assign more experimental MS peaks, identify additional peptide fragments (and corresponding proteins), and reduce the complexity of the MS data set further. The process can be performed in an iterative manner until no further unidentified MS peaks can be assigned. Depending upon the protein complement of the sample, this iterative process can be used to identify 50%, 75%, 90%, 95%, 99% or essentially 100% of the member proteins of the sample.

As another embodiment of the present invention, method of reducing a number of peaks to further be analyzed in a mass spectrum for a sample are provided. The methods include the steps of a) generating a first amino acid sequence database comprising an amino acid sequence of at least one protein present in the sample; b)calculating a first list of theoretical masses for a first set of known in silico proteolytic peptides generated from the first database; c) correlating a first theoretical mass with a position of an unidentified MS peak in a mass spectrum for the sample, thereby determining the presence in the sample of a first protein that comprises a peptide having a mass equal to the first theoretical mass; and d)identifying one or more MS peaks that correspond to masses for the known in silico proteolytic peptides, thereby reducing the number of peaks to further be analyzed in the mass spectrum.

Reducing Complexity Due to Redundant Peptides from Identified Proteins

As another optional aspect of reducing data complexity, additional MS peaks derived from an identified protein can be removed from the experimental mass list without direct identification. This can be achieved by i) calculating theoretical molecular masses for additional in silico polypeptides derived from an identified protein; and ii) analyzing the mass peak list of MS data and assigning the mass peaks to the identified protein (i.e., removing the mass spectral data from further analysis) if the mass spectral data for the additional peptide meets certain strict criteria. As a first criterion, the mass peak in question can be removed from further consideration (e.g., disregarded due to putative assignment) if a) the mass peak is within 5 ppm mass tolerance (or 4 ppm, or 3 ppm, or 2 ppm, or 1 ppm, depending upon the stringency desired) of the theoretical molecular mass of an additional in silico peptide derived from the previously identified protein. Optional additional criteria include if either b) the expression ratio determined for the additional peptide corresponds to an expression ratio for the first identified peptide; and/or c) the second peptide contains the expected number of derivatized amino acids (i.e., the observed number of selected amino acids, as determined by isotope labeling, corresponds to the number of expected theoretical derivatized amino acids for the second in silico peptide.) This procedure can also be used in alternative steps of the methods of the present invention, e.g., as an aspect of correlating the theoretical masses for the identified proteins with unidentified members of the mass peak list.

As an example, after a particular proteolytic peptide has been identified using the accurate mass techniques of the present invention (or optionally after identification by another method, such as tandem MS), the sequence of the identified parent protein is used to generate a list of additional in silico peptides and corresponding theoretical masses. For samples that were derivatized using an amino acid-specific or functional-group specific derivatizing agent, the list of additional in silico peptides can be limited to include only those fragments containing the appropriate number of selected amino acid constituents. Furthermore, larger polypeptides having “missed” proteolytic cleavages can also be included in the list of additional in silico peptides. During data analysis, every MS peak in the mass list that matches (i.e. corresponds to) a mass of an additional in silico proteolytic fragment (e.g., that is within the 5 ppm, or 4 ppm, or 3 ppm, or 2 ppm, or 1 ppm mass tolerance, depending upon the selected criteria) can be assumed to be from the identified parent protein and can be removed from further consideration. Comparison of the expression ratios for the originally-identified peptide and putatively identified (i.e. additional) peptides, and confirmation that the expected number of selected amino acids are present (based upon isotope labeling data in the mass spectrum) can be used an additional assurance that the peak has been correctly identified. While it is possible, although unlikely, that a peptide from another protein will also be removed from consideration even with stringent criteria, yet-to-be identified protein that produced the mis-assigned peptide will also produces tens of other peptides that will ultimately allow it to be identified.

As another aspect of the present invention, the methods include the steps of a) contacting a sample that comprises a plurality of proteins with at least a first proteolytic reagent that cleaves proteins at defined cleavage sites to form sample proteolytic peptides; b) contacting the sample with at least a first derivatizing agent that specifically labels a selected amino acid (or a specific functional group) when the selected amino acid is present in a sample protein; c) determining a first mass for a first proteolytic peptide; d) comparing the first mass to theoretical molecular masses for a plurality of in silico proteolytic peptides that are derived from amino acid sequences for a plurality of proteins, wherein a match between the mass determined for the first proteolytic peptide and the theoretical molecular mass for an in silico proteolytic peptide is indicative of the presence in the sample of the protein from which the in silico proteolytic peptide is derived; e) calculating theoretical molecular masses for additional in silico proteolytic peptides derived from the protein identified in the comparison of the mass determined for the first proteolytic peptide to the theoretical molecular masses; and f) analyzing a mass spectrum (or set of mass spectra) generated using a mass spectrometer that provides a mass accuracy of 5 ppm or better for additional MS peaks that correlate to (e.g., are within 5 ppm of) the theoretical molecular masses for the additional in silico proteolytic peptides, thereby assigning these peaks to the previously-identified protein and disregarding the mass spectral data from further assignment consideration. Optionally, the method further includes determining an expression ratio for the first proteolytic peptide, wherein the mass spectral data for the second proteolytic peptide is disregarded if the mass spectral data a) is within 5 ppm (or 3 ppm or 1 ppm) of the mass of an in silico peptide, and if either b) if the expression ratio determined for the second peptide corresponds to the expression ratio for the first peptide and/or c) the number of derivatized amino acids (or functional groups) of the second peptide corresponds to the number of theoretical derivatized amino acids or functional groups for the second in silico peptide.

Reducing Complexity Due to PTM Peptides from Identified Proteins

Mass measurements at 5 ppm accuracy (or better), CRAMP, and optional tandem MS confirmation can be used as described herein for protein identification, by comparison of experimental mass values against those expected from various protein and/or genome database sequences. However, in cellular systems, the active forms of protein are often different than what is predicted from the sequence of a gene. Genomic sequence databases contain little information about the specific post-translational modifications (PTMs) of the member proteins (e.g., glycosylation, phosphorylation, sulfation, fatty acid attachment, and the like), beyond the presence or absence of a known amino acid motif typically associated with the PTM. Proteomic samples contain the information, but is typically harder to decode. The presence of post-translationally modified peptide sequences in the sample generates a subset of experimentally determined masses that do not match any of those calculated in silico based upon the sequence alone, leading to unassigned peaks in the mass spectrum. The methods of the present invention can also be employed to identify peptides having PTMs or other irregularities in amino acid sequence (e.g., non-standard amino acids, chemical modifications, etc.)

Despite post-translational modifications, a large number of proteolytic peptides from any given protein will still be identified by the initial steps performed during the accurate mass and CRAMP analysis, because the sample proteolytic peptides also span regions of the protein sequence that remain unmodified. Thus, while not all of the mass spectra peaks will have been assigned after the first iteration of the methods of the present invention, the database of identified proteins generated will likely still represent the majority of proteins present in the sample. Assuming that all of the proteins present have been identified and their related (unmodified) proteolytic fragments assigned to MS peaks, the remaining unassigned masses from, for example, a multidimensional LC/MALDI FT-ICR experiment, will deviate from those in the database exactly by the one or more post-translational modifications that occur on those peptides. These can then be assigned by the additional analysis steps of the methods of the present invention.

As an additional aspect of identifying any remaining unassigned MS peaks, correlating the first list of theoretical masses from the identified proteins with unidentified members of the mass peak list of experimental mass peaks optionally includes, but is not limited to, the steps of: a) selecting a type of peptide modification to be considered during the next iterative step; and b) generating theoretical masses for the first set of in silico proteolytic peptides generated from the first database, wherein member proteins are assumed to contain one or more occurrences of the peptide modification. For the purpose of generating the theoretical masses, the identified sample proteins provided in the first database are assumed to contain one or more of the selected peptide modification(s), optionally based upon the amino acid motif typically present for the selected.

Any number of peptide modifications (both reversible and irreversible) can be considered in the methods of the present invention, including, but not limited to, phosphorylation, fatty acids esterification (e.g., myristoylation, glycophospatidylinositol-anchoring), N-linked and O-linked oligosaccharides, ADP-ribosylation, methylation or acetylation, and the like. In addition, other mass altering peptide modifications, such as chemical modifications (e.g., acetylation, deamination), affinity labeling, isotope labeling, or amino acid substitutions with, for example, non-standard (a typical) amino acids are also considered. Putative positions of the modification on proteins in the first or second databases can be generated, for example, using computer algorithms for predict potential protein post-translational modifications based upon known amino acid motifs. One exemplary program for this purpose is FindMod available online via the Expert Protein Analysis System (ExPASy) proteomics server of the Swiss Institute of Bioinformatics (http://ca.expasy.org/tools/).

An interesting feature of many of these post-translational modifications is their “mass defect” (see, for example, Lehmann et al. (2000) “The information encrypted in accurate peptide masses: Improved protein identification and assistance in glycopeptide identification and characterization” J. Mass Spectrom. 35:1335-1341). All possible peptide compositions (without post-translational modifications) exhibit a gaussian-shaped profile of masses for every given nominal monoisotopic mass M with a center of the distribution at an approximate mass Mp=M+0.00048M Da with a total width that encompasses 95% of all possible peptides Wp=0.19+0.001M Da (Zubarev et al. (1996) “Accuracy Requirements for Peptide Characterization by Monoisotopic Molecular Mass Measurements” Anal. Chem. 68:4060-4063). The mass defect for many of these post-translational modifications will significantly shift this distribution to either the high or low mass side, depending on the modification. For example, a phosphate group added to a peptide with the centroid mass for M=1000 results in a mass of 1080.44635, but the centroid mass for M=1080 should actually be 1080.5184, indicating that phosphorylation induces a downward shift of over 0.07 Da for the peptide distribution. On the other hand, the attachment of a myristoyl group (mass 210.19836) to the centroid mass for M=1000 results in a peptide with mass 1210.67836 versus a centroid mass for M=1210, 1210.5808, indicates an upward shift in centroid mass of almost 0.10 Da. Rejecting putative assignments for data having an unexpected shift in mass for the distribution of peptide masses reduces the likelihood that a modified peptide will be incorrectly identified (since the peaks will not match an unmodified peptide within 1 ppm mass), particularly when combined with additional criteria such as the same sequence characteristics (same number of lysines, acidic amino acids, cysteines, etc.). Optionally, the identity of the post-translationally modified polypeptide is confirmed by additional experimentation, such as performing tandem MS on the sample peptide.

“Accurate Mass” Platform

In peptide mapping experiments, sequence specific proteases or certain chemical agents are used to obtain a set of peptides from the sample protein that are then mass analyzed. The observed masses of the proteolytic fragments are compared with theoretical “in silico” digests of all the proteins listed in a sequence database. The matches or “hits” are then statistically evaluated and ranked according to the highest probability. Based on the mass accuracies afforded by typical mass spectrometers, matching 5-8 different tryptic peptides is usually sufficient to unambiguously identify a protein with an average molecular weight of 50 kDa. Although simple to implement, the technique assumes that all the masses arise from a single protein, making the identification of proteins that exist in a mixture very difficult.

By contrast, the ability to obtain mass measurements with extremely high accuracies can lead to the identification of a protein based on the measurement of a single peptide if it has a mass unique from all other possible in silico generated fragments. This information is sometimes supplemented by partial knowledge of the amino acid composition of the measured peptide (e.g., as elucidated through chemical labeling strategies), the proteolytic enzyme or chemical used, etc. Since identification can be made on the basis of a single peptide, high mass accuracy protein identifications can combine the unique operational advantage of LDI analyses with the ability to identify proteins from complex mixtures without exhaustive prefractionation.

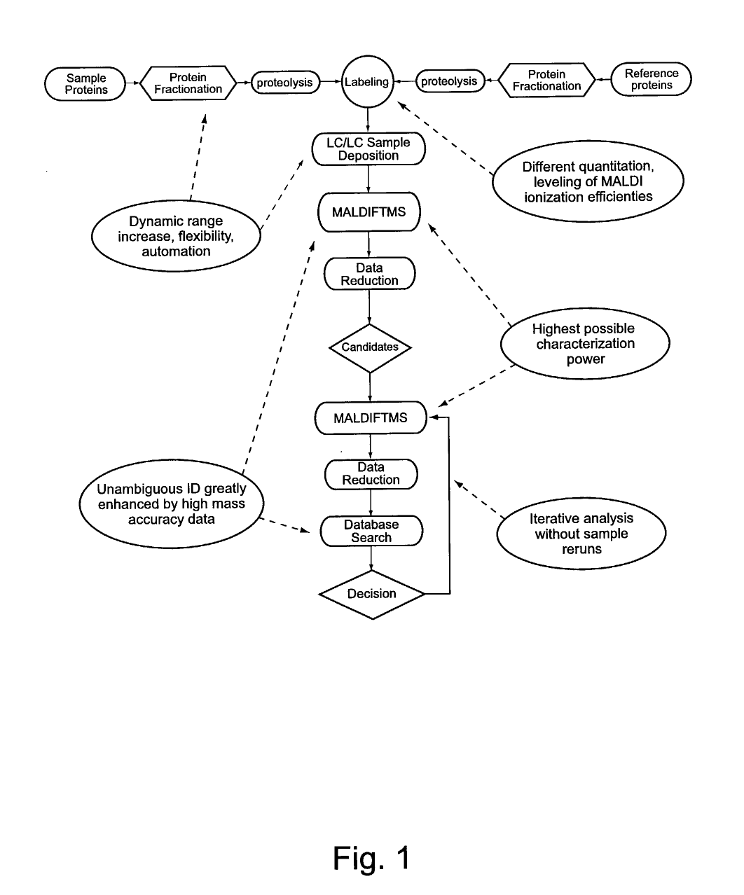

The present invention provides methods for identifying two or more proteins in a sample using LDI-MS. A flowchart depicting one embodiment of the steps in an exemplary “accurate mass” analysis platform is provided in FIG. 1. Although the chart outlines the experimental flow of a differential display-type experiment, comparable analytical procedures can also be used for other studies, including peptide mapping, determination of the constituents of protein complexes, PTM identification, and time-course studies.

In one aspect of the present invention, methods of protein identification using “unique” masses are provided. The methods include, but are not limited to, the steps of a) providing a sample comprising a plurality of proteolytic polypeptides; b) ionizing member polypeptides by LDI and obtaining a mass of at least a first polypeptide using a mass spectrometer that provides a mass accuracy of 5 ppm or better; c) comparing the mass of the first polypeptide to members of a database of theoretical molecular masses for a plurality of in silico proteolytic peptides, wherein each member in silico peptide has a unique theoretical mass, and wherein a match between the mass obtained for the first polypeptide and the unique theoretical mass for an in silico proteolytic peptide indicates that a parent protein comprising the in silico polypeptide is present in the sample, thereby identifying a first protein in the sample; and d) repeating the comparing step for one or more masses obtained for additional sample polypeptides, thereby identifying additional proteins in the sample.

In an additional embodiment of the present invention, the methods include the steps of a) contacting a sample containing a plurality of proteins with a first derivatizing agent, wherein the first derivatizing agent comprises at least two isotopic forms and specifically labels a selected amino acid or functional moiety when the selected amino acid is present in a sample protein; b) fractionating the sample and depositing a plurality of fractions of an eluent onto a solid support suitable for laser desorption/ionization (LDI) MS; c) ionizing member polypeptides (e.g., at least a first polypeptide) in one or more of the fractions by LDI and obtaining a mass of the polypeptide using a mass spectrometer that provides a mass accuracy of 5 ppm or better; and d) comparing the mass obtained for the polypeptide to members of a database of unique theoretical molecular masses for a plurality of in silico proteolytic peptides that are derived from amino acid sequences for a plurality of proteins; wherein a match between the mass obtained for the polypeptide and the theoretical molecular mass for an in silico proteolytic peptide is indicative of the presence in the sample of the protein from which the in silico proteolytic peptide is derived, thereby identifying a first protein in the sample. Optionally, the method also includes an iterative aspect, by repeating the comparing step for one or more masses obtained for additional polypeptides, thereby identifying additional proteins in the sample.

Optionally, the protein identification methods as described further include cleaving or fragmenting the sample proteins into polypeptide fragments, either before or after the labeling/derivatization step. For example, in yet a further embodiment, methods are provided for analyzing MS peaks from a proteomic sample, including the steps of: a) contacting a sample having a plurality of proteins with at least a first proteolytic reagent that cleaves proteins at defined cleavage sites to form sample proteolytic peptides; b) contacting the sample with at least a first derivatizing agent that specifically labels a selected amino acid or functional group when the selected amino acid or functional group is present in a sample protein; c) subjecting at least a first proteolytic peptide to mass spectrometry to determine a mass of the first proteolytic peptide; d) comparing the mass determined for the first proteolytic peptide to unique theoretical molecular masses for a plurality of in silico proteolytic peptides that are derived from distinct amino acid sequences for a plurality of proteins (wherein a match between the mass determined for the first proteolytic peptide and the unique theoretical molecular mass for an in silico proteolytic peptide is indicative of the presence in the sample of the protein from which the in silico proteolytic peptide is derived); e) calculating theoretical molecular masses for additional in silico proteolytic peptides derived from the protein identified in the comparison of the mass determined for the first proteolytic peptide to the theoretical molecular masses; and f) subjecting at least a second proteolytic peptide to further mass spectrometry, and disregarding mass spectral data for the second proteolytic peptide if the mass spectral data matches that which would be obtained for one or more of the additional in silico proteolytic peptides from the previously identified protein (e.g., is within 5 ppm, preferably within 2 ppm, more preferably within 1 ppm).

The details regarding the methodology, as well as systems for performing the methods of the present invention, are provided in greater detail below. Before describing the present invention in detail, it is to be understood that this invention is not limited to particular populations of protein sequences or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a derivatization agent” includes a combination of two or more agents; reference to “a polypeptide” includes mixtures of polypeptides, and the like.

Samples for Analysis

Any number of samples can be examined and the constituent proteins identified using the methods of the present invention. One advantage to these methods is that, optionally, the methods can be used to identify at least 50%, at least 75%, at least 85%, at least 90%, at least 95%, at least 99%, or essentially all (100%) of the constituent proteins in the sample.

As such, the methods and systems of the present invention are particularly useful in analyzing proteome samples. A “proteome” is, in simplest terms, the protein complement expressed by a genome. The proteome can be derived from a human genome, a yeast genome, a Drosophila genome, a bacterial genome, or other organism of interest. Optionally, the sample comprises a “sub-proteome,” e.g., a portion or subset of the proteome. Exemplary sub-proteomes of interest include, but are not limited to, the proteins involved in a selected metabolic pathway (for example, glycolysis, lipogenesis, polyketide synthesis, or signal transduction), or a set of proteins having a common enzymatic activity (G-protein receptors, protein kinases, and the like). For example, preparations of organelles, ribosomes, or protein complexes can be analyzed using the provided methods and integrated systems. While simple mixtures of proteins can be examined using the methods of the present invention, one strength of the invention is in the ability to analyze and identify components of a plurality of proteins having at least 50 constituents, or preparations of at least 100 constituent proteins, or preparations of at least 1,000 proteins, or even complex populations of tens of thousands of constituents (for example, 10,000 proteins, 15,000 proteins, 20,000 proteins or 25,000 proteins).

Isotopically-Labeling Sample Peptides

The methods and systems of the present invention are based upon being able to accurately measure masses, such as the mass of an isotopically-labeled polypeptide. A match between the mass obtained for the polypeptide and the theoretical molecular mass for an in silico polypeptide is indicative of the presence in the sample of the protein from which the in silico polypeptide is derived. Therefore, the sample peptides need to be labeled in a highly selective and reproducible manner, and the masses of the resulting isotopically-tagged molecules must be accurately determined.

In some embodiments of the present invention, the methods include the step of contacting a sample that comprises a plurality of proteins with a first derivatizing agent, wherein the first derivatization agent comprises at least two isotopic forms and specifically labels a selected amino acid or functional moiety when the selected amino acid or functional moiety is present in a sample protein. The derivatizing agent is a chemical entity that is capable of binding and specifically labeling a select amino acid (e.g., lysine, cysteine), or a or functional moiety, or particular type of amino acid (e.g., acidic, basic, aromatic), when the selected amino acid is present in a sample protein or polypeptide.

In an alternative embodiment of the present invention, proteins in the sample are labeled in situ by providing a cell with the isotopically-labeled derivative agent. For example, cells can be grown in isotopically-labeled media components (e.g., an isotopically-labeled amino acid precursor), thereby labeling the proteins in situ. Thus, both chemical derivatization methods and in situ labeling methods are contemplated in the methods of the present invention.

The derivatizing agent is typically provided in two isotopic forms, in order to facilitate identification of the derivatized polypeptides. The sample proteins are contacted with the different isotopic versions of the same reagent (either in separate reactions or in a single pooled reaction). The result is a series of isotopically labeled polypeptide pairs, with the relative concentration of each member of a given pair being directly proportional to its signal intensity. For example, an amino acid-specific derivatization agent is provided in two isotopic forms, e.g. a deuterated version and a non-deuterated version. The proteins derivatized with this agent will be present in a mixture of deuterated and non-deuterated forms based upon the number of selected amino acids (or functional moieties which interact with the agent) in the polypeptide and the extent of labeling (e.g. percentage of total moieties labeled).

In embodiments in which the number of occurrences of a specific amino acid (or a type of amino acid or a chemical functionality) is desired, the sample can be labeled with fixed amounts (typically, but not necessarily, equimolar) of both forms isoforms. Alternatively, the isotopic labels can be used in differential quantitation experiments, in which two (or more) different samples are labeled with different isotopic forms, and recombined. In this embodiment, differences in peak heights between two members of a pair represents the change in concentration of that species between the two samples. These and other labeling embodiments are contemplated for use in the methods of the present invention.

While deuteration is a common isotopic form for use in the methods of the present invention, isotopes of other atoms are optionally employed. For example, bromine is naturally present as a 50:50 ratio of ⁷⁹Br and ⁸¹Br; thus, bromine-labeled derivatizing agents inherently comprise a mixture of the two isotopes. Additional exemplary isotopes for use in the methods of the present invention include, but are not limited to, ¹³C, ¹⁴C, ¹⁵N, ¹⁸O, ³⁵Cl, and ³⁷Cl labeled agents. While unstable isotopes (e.g., radioactive-labeled compounds) are not commonly examined by MS, these labels can also be employed in the methods of the present invention. Preferably, the derivatizing agent is specific for the amino acid(s) to be labeled, and will not extensively cross-react with alternative moieties (e.g., N-terminal amino groups, or C-terminal carboxyl groups).

In some embodiments, the isotopic forms are provided in “natural” proportions, for example, when using bromine-labeled agents. In other embodiments, the derivatizing agents comprise unnatural isotopic proportions of one or more stable isotopes, which can be selected or adjusted depending upon the experiment performed. Any isotopic variations of the derivatizing agents can be used the present invention, whether stable or not, and are intended to be encompassed within the scope of the present invention. Optionally, three or more isotopic forms of the derivatizing agent can be used in the methods and with the systems of the present invention, with the appropriate adjustments made for the analysis of the resulting multiple products.

Which amino acid or functional group is selected for labeling will differ with the selection of sample and availability of specific derivatizing agents and can easily be determined by one of skill in the art. For example, lysine resides can be labeled by any of a number of chemical reagents, including, but not limited to, succinic anhydride and disuccinimidyl suberate. However, reagents that derivatize to the basic side chain of lysine residues might also bind to the N-terminal group of the polypeptide in a non-selective manner. Optionally, the derivatizing agents are chosen and/or the reaction conditions are adjusted such that the selected derivatizing agent reacts with less than 10%, and preferably less than 1%, of the nonselected (e.g. N-terminal amino) groups. One preferred labeling agent for use in the methods and systems of the present invention is 2-methoxy-4,5-dihydro-1H-imidazole, a reagent used to specifically label lysine residues (see, for example, U.S. Ser. No. ______ (GNF docket No. P0051PC30) titled “Labeling Reagent and Methods of Use” co-filed herewith). In addition to specifically labeling lysine sidechains, this reagent also increases the ionization efficiency of the lysine-containing peptides.

The derivatizing agent 2-methoxy-4,5-dihydro-1H-imidazole reacts with the amino group of a lysine residue to form its 4,5-dihydro-1H-imidazol-2-yl derivative. Peptide mapping experiments of tryptic protein digests after reaction with this reagent suggest that total amino acid sequence coverages is nearly doubled as compared to that of the unlabelled counterparts (Peters et al. (2001) Rapid Commun. Mass Spectrom. 15:2387-2392). In addition, isotopic substitution of deuterium at the two methylene ring carbons simultaneously enables differential quantitation by affecting a 4 Da mass difference per labeled lysine. Other mass differences can also be affected by performing different functionalization reactions at these two ring positions. This additional compositional information generated by differential labeling of the sample can greatly simplify the database search required to identify the protein from which a given peptide is derived.

Another preferred class of derivatizing agents are cysteine-reactive compounds. There are thousands of cysteine selective labels which can be used in the methods of the present invention. The thiol-reactive functionality of the cysteine sidechain, being a good nucleophile and mild oxidizing agent, can rapidly react in different manners to produce a covalent bond. Thus, thiol-reactive functionalities generally are reactive electrophiles. Three general classes of cysteine-selective labels include haloacetyls, maleimides, and disulfide bond forming reagents.

The haloacetyl compounds typically fall under the general chemical structure ROOCCH₂X, where X=I or sometimes Br, and R can be any alkyl group. Variations in the isotopic content of the alkyl group can give rise to numerous stable isotope pairs, in addition to the natural isotopic content of Br. A classic example of a haloacetyl-type cysteine labeling reagent is iodoacetamide; a popular alternative zwitterionic derivative is S+2-amino-5-iodoacetamido-pentanoic acid. In addition, the commercially available ICAT (isotope coded affinity tag) labels generally are compounds of this category (see, for example, Gygi et al., supra).

Michael acceptors such as maleimide, acid halides, and benzyl halides also are good cysteine labeling derivatizing agents. The maleimide-type labels are unique Michael acceptors for cysteine. Structurally, these reagents are ring compounds having an R group attached, allowing for multiple isotope substitution possibilities. One exemplary maleimide-based derivatizing agent is N-ethyl maleimide.

The ability of the free sulfhydryl group to form disulfide bonds offers another approach ability to label cysteine-containing proteins The free sulfhydryl of the cysteine residue can be reacted with a disulfide of a derivatizing agent, such that the interaction is converted to a disulfide bond. This reaction is reversible, and can be used to regenerate the original sulfhydryl group. Hundreds of derivatizing agents fall under this category and are available for use by one of skill in the art, including a reversible ICAT analog.

Finally, cysteine residues can be labeled using vinylpyridines (e.g., 4-vinylpyridine), as described in, for example, Ji et al., supra.

Additional derivatizing agents include reagents that label carboxyl groups (such as Woodward's reagent K, carbodiimides, epoxides, diazoalkanes, diazoacetates, and esterification using methanolic HCl), amino groups (O-methylisourea, succinic anhydride, N-hydroxysuccinimide derivatives), histidine imidazole groups (diethylpyrocarbonate), and tyrosine side chains (N-acetylimidazole, tetranitromethane). Thus, potentially any derivatizing agents known or designed by one of skill in the art can be used in the methods of the present invention.

In one embodiment of the methods, the sample is divided into two (or more) portions. A first portion of the sample is contacted with the first isotopic form of the derivatizing agent, the second portion of the sample is contacted with the second isotopic form of the agent, etc. Once labeled, the sample portions are recombined prior to further analysis. In an alternative embodiment, the isotopic forms of the derivatizing agent are provided as a mixture prior to contacting the sample (for example, as with the case of bromide-labeled compositions).

Furthermore, the labeling of the sample proteins via the derivatizing agent can be performed at any time prior to ionization of the sample fractions. Optionally, the sample and the derivatizing agent are contacted prior to fractionation, although derivatization could also be performed upon the eluted fractions. Furthermore, the derivatizing agent can be reacted with the sample either prior to or after the optional cleaving of the sample, as described below.

Instrumentation

Another important aspect to the methods of the present invention is in the selection of instrumentation employed in both the ionization as well as the mass measurement step. In particular, the high resolution, mass accuracy, and dynamic range of Fourier transform ion cyclotron resonance (FT-ICR) MS systems are particularly suitable for the methods and integrated systems of the present invention.

The high mass accuracy mass spectrometer used in the present invention is capable of providing a mass accuracy of 5 ppm or better. Optionally, the mass spectrometer provides a mass accuracy of 4 ppm or better, 3 ppm or better, 2 ppm or better, or 1 ppm or better). Not only do high mass accuracy measurements provide greater confidence in protein identification assignments, but they also enable proteins to be identified with either less sequence coverage (in the case of peptide mapping) or fewer additional tandem MS experiments. High mass measurement accuracy optionally allows protein identifications to be made on the basis of the mass of a single peptide, providing higher-throughputs in the analysis of mixtures due to the significant decrease in time spent on additional tandem MS experiments. In addition, a concomitant time saving in the cross correlation process of mass spectral data with in silico digested databases would also be achieved.

In a preferred embodiment, the methods and systems of the present invention employ a Fourier-transform ion cyclotron resonance mass spectrometer (FT-ICR MS). FT-ICR mass spectrometers provide an unparalleled mass accuracy (˜1 ppm), high resolution (routinely>100,000), large dynamic range (routinely 10³ and possibly 10⁴), and good sensitivity (amol). The methods and systems of the present invention are designed to leverage the full advantages of FT-ICR MS within an automated, robust analysis platform.

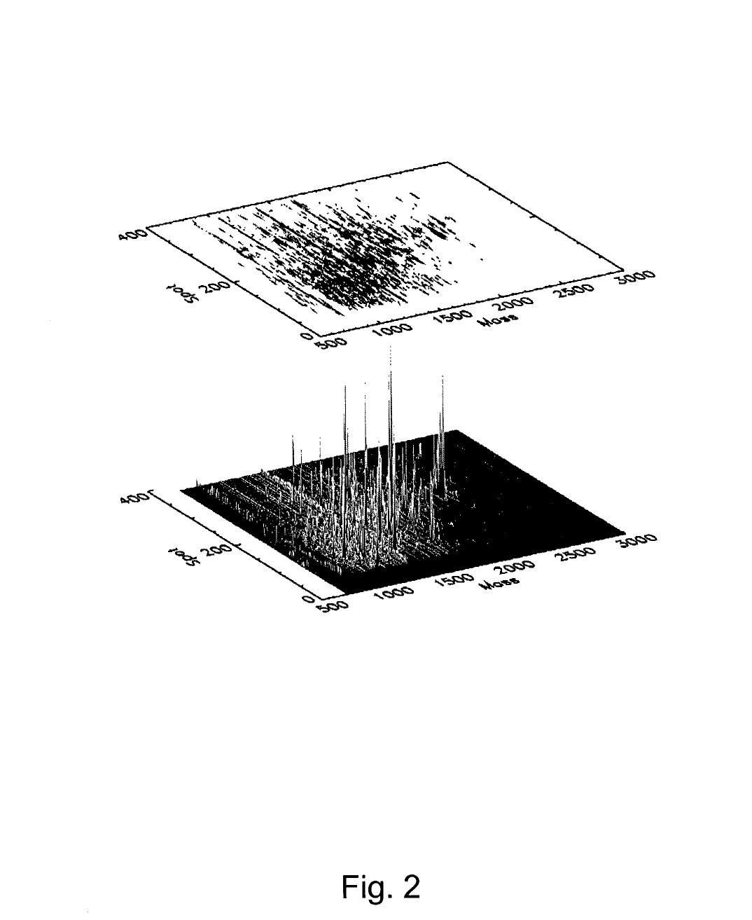

Some embodiments of the methods of the present invention were performed using a modified 7.0 T Bruker Apex II FT-ICR instrument, equipped with a home-built MALDI source, a new open-cylindrical cell, and a quadrupole mass spectrometer (ABB Extrel). Replacement of the originally installed cell with a larger capacitively-coupled open cylindrical cell improved the dynamic range an order of magnitude (from ˜10³ to ˜10⁴). For comparison, a digest of yeast cytosolic proteins was reverse-phase separated and 10 seconds fractions were spotted directly onto a MALDI plate. Using the originally supplied cell, 3,000 individual peptides were resolved while over 10,000 could be resolved with the newer cell (see FIG. 2).

Optionally, an electrospray spectrometer can be used in the methods of the present invention. However, the “permanent record” obtained by deposition of a separation column's eluent onto an LDI target plate provides several advantages compared to a real time coupling of the separation method and an electrospray ionization mass spectrometry (see, Griffin T J et al. (2001) Anal. Chem. 73:978). Implementation of an electrospray-based ionization protocol using sample fractions collected and stored on a solid support is contemplated in the present invention, but not a preferred embodiment.

Proteolytic Cleavage of Sample Proteins

In most embodiment of the present invention, the sample proteins are contacted with a proteolytic reagent that cleaves proteins at defined cleavage sites, thereby generating the sample proteolytic polypeptides. This proteolytic step can be performed either prior to or after contacting the sample with a derivatizing agent. Optionally, the cleaving of sample proteins can even be performed after fractionation of the sample.

Proteolytic reagents for use in the methods of the present invention include both proteolytic enzymes as well as chemical cleavage reagents. In one embodiment of the present invention, the proteolytic reagent is selected from proteolytic enzymes such as of trypsin, chymotrypsin, endoprotease ArgC, aspN, gluC, and lysC (or combinations thereof can be used). The enzymes, as well as any additional enzymes not specifically listed, can be used alone or in combination to generate proteolytic fragments of the sample proteins.

Alternatively (or in combination with the enzymatic approach), the proteolytic reagent can include a chemical cleavage reagent, such as cyanogen bromide, formic acid, or thiotrifluoroacetic acid. Optionally, the sample can also be treated to remove post-translational modifications or other mass-altering moieties, prior to subjecting the proteolytic peptides to mass spectrometry.

Optionally, the methods of the present invention include the step of selecting a subset of cleaved peptides of a desired size range. For example, subsets of peptides having greater than 5 amino acids, greater than 10 amino acids, greater than 25 amino acids, and the like, can be selected for analysis. The selection can be performed, for example, by restricting size ranges to be analyzed by mass spectrometry, or by performing a size fractionation procedure prior to MS analysis.

In an alternate embodiment, the sample proteins comprise truncated polypeptide sequences. The peptides can be truncated due to, e.g., DNA mutagenesis, interrupted synthesis, or due to post-translational proteolysis. Optionally, theoretical masses are calculated for in silico peptide sequences representing various possible position of truncation for a peptide having n amino acids (e.g., aa₁-aaₙ₋₁, aa₁-aaₙ₋₂, where n represents the total amino acids in the peptide) as well as varying the position of the first amino acid of the in silico peptide (e.g., aa₂-aaₙ, aa₃-aaₙ, etc.) or combinations thereof (aa₂-aaₙ₋₄). The truncation alternatives selected for generating the in silico peptide sequences and related list of theoretical masses will depend in part upon the sample being examined and can be selected as such.

Fractionation of the Sample

The protein identification methods of the present invention do not require a physical simplification of the sample prior to collecting the mass spectral data; thus, data collection optionally can be performed without further fractionation of the plurality of proteins (or data from multiple spectra can be tabulated into a master list of MS peak positions and analyzed together). This is in contrast to the current MS approaches to proteome analysis, such as the ICAT strategy (Gygi et al., supra) where, at most, only a few peptides per protein are present in the mixture analyzed by the mass spectrometer. Since each fraction might contain tens to hundreds of peptides derived from the same protein, identification will be attempted for all of these peptides (at a rate of a few peptides at a time) using the methods currently available in the art.

In the methods of the present invention, having multiple peptides generated from a particular protein is advantageous in that the redundant information provides multiple opportunities to unambiguously identify the particular protein. However, after that identification is obtained, this information then becomes a hindrance, leading to redundant information and a significant reduction in throughput. The data complexity reduction methods of the present invention can optionally be employed with the protein identification methods, thereby providing an (optionally iterative) mechanism for addressing the redundancy in proteomics MS data (or other large MS data sets) as described above.

In the methods of the present invention, fractionating the sample includes any of a number of one-dimensional as well as multi-dimensional techniques known to one of skill in the art, including, but not limited to, performing liquid chromatography (LC), reverse phase chromatography (RP-LC), size exclusion chromatography, ion exchange chromatography, affinity chromatography, capillary electrophoresis, gel electrophoresis, isoelectric focusing, and the like. Another technique which can be used is immobilized metal ion affinity chromatography (IMAC), as described in, for example, Porath (1992) “Immobilized metal ion affinity chromatography” Protein Expr Purif 4:263-81; and Cao, supra.

Electrophoretic methods of separation can also be used to fractionate the sample. For example, capillary electrophoresis, ID or 2D gel electrophoresis, isoelectric focusing, or other electrophoretic methods can be employed. Furthermore, combinations of these and other separation methodologies can be used to fractionate the sample into portions for analysis by mass spectrometry.

The plurality of fractions generated during the fractionating step can be generated either by “sampling” portions of the eluent, or preferably, by deposition of the eluent directly onto the solid support for analysis. In a preferred embodiment, depositing the plurality of fractions is accomplished using an automated dispensing system. A suitable deposition system is described in International Patent Application No. PCT/US02/01536, filed Jan. 17, 2002. Specialized liquid junction-coupled sub-atmospheric pressure deposition chambers for the off-line coupling of capillary electrophoresis with MALDI MS have also been described (see, for example, Preisler et al. Anal. Chem. 1998, 70, 5278-87 and Preisler et al. Anal. Chem. 2000, 72, 4785-95).

The eluent generated during the final fractionation step is deposited or spotted (in the form of a plurality of fractions) onto a solid support suitable for mass spectrometry. Typically, the solid support comprises a surface modified for sample confinement, such as a plate containing structural confinement elements (e.g., wells or depressions), chemical modifications which induce sample localization (e.g., hydrophilic or hydrophobic regions), and the like. Preferably, solid support comprises a hydrophobic/hydrophilic MS source plate.

The performance of LDI-type experiments such as MALDI MS can greatly be affected by competitive ionization effects, which are especially prevalent in complex mixtures (such as proteomic samples). In a preferred embodiment, micro high performance liquid chromatography (HPLC) is employed as a final fractionation step. The reversed-phase separation technique, in combination with an automated deposition system as described herein and in U.S. Ser. No. ______ [Attorney Docket No. 36-003010US] minimizes these effects by providing a reproducible environment for the recrystallization of matrix and analytes with similar hydrophobicities. Additionally, the deposition system works equally well with aqueous or numerous organic solvents, enabling both on-plate recrystallization processes not limited to solvent mixtures of acetonitrile and water, as well as the use of matrices such as alpha-cyano-4-hydroxycinnamic acid (HCCA) that are typically incompatible with anchor plate technology.

Optionally, the methods for protein identification as provided by the present invention further comprise the steps of identifying one or more fractions that contain a proteolytic peptide for which no unambiguous match was observed among the in silico proteolytic peptides; and subjecting that fraction to further analysis to identify the proteolytic peptide that is present in the fraction. Further analysis of the fraction can be performed, for example, by tandem mass spectrometry.

Preparation of Fractionated Samples

In some embodiments of the present invention, the sample fractions are deposited upon a support suitable for performing LDI. Optionally, the sample fractions can be collected via an alternative collection system (e.g., microtiter wells or the like); aliquots of the eluted fractions are then transferred to the LDI-suitable platform or otherwise prepared for ionization. As noted previously, deposition of a separation column's eluent onto a solid support prior to mass spectral analysis provides several advantages compared to a real time coupling of the separation method and mass spectrometer.

The solid support used in the methods and devices of the present invention typically comprise a surface modified for sample confinement. For example, the solid support can be a surface having one or more wells, channels, indentations, raised walls, or the like. In addition or alternatively, the surface of the solid support is modified chemically to effect sample localization in particular regions of the surface (e.g., hydrophilic or hydrophobic regions, affinity-labeled regions, and the like). Preferably, the solid support comprises a hydrophobic/hydrophilic MALDI plate. U.S. patent application Ser. No. ______ [Attorney Docket No. 36-006810US] titled “Sample Preparation Methods for MALDI Mass Spectrometry” co-filed herewith provides additional methods related to sample preparation for MS analysis which can be employed in the methods of the present invention. For example, methods for co-crystallizing sample fractions with LDI-suitable matrices in the presence of MALDI-incompatible (e.g., non-standard) solvents are provided. In addition, a procedure for internal calibration involving premixing of the sample and calibrant prior to mass detection is also provided.

With respect to sample fractionation, the sample fractions can be deposited directly onto a target plate. In one embodiment, the outlets of a series of μHPLC columns are arranged in parallel, and MALDI target plates positioned on an x,y translational stage are automatically moved underneath the columns. The effluents of the columns are transferred to the plates through a charge induction mechanism by applying an intermittent negative potential to the plates, resulting in a series of droplets of precisely controlled volume.

Preferably, specially-patterned target plates consisting of hydrophilic anchors or “target regions” arrayed on an otherwise hydrophobic surface are used to collect the sample fractions (see, for example, Schuerenberg et al. (2001) Anal. Chem. 72:3436-3442). After deposition of a sample onto an anchor, both the analyte and matrix localize into an area smaller than that occupied by the original droplet as the solvent evaporates, resulting in concentration of the analyte. The use of such target plates provides considerable advantages. For example, the sensitivities of ESI methods are known to be concentration dependent, often necessitating the use of nanochromatography to achieve maximum sensitivity. Although effective, such nanoscale chromatography systems present practical problems and often require the manual loading of samples directly onto the separation column. By contrast, the anchor target plates further concentrate the samples after the chromatographic process is complete, enabling the use of 300 μm internal diameter (id) capillary columns and commercial autosamplers. Localization of analytes to precisely defined locations approximately 400 μm in diameter enables the MALDI stage to rapidly query only those regions that contain analyte. In addition, increasing the size of the area irradiated by the MALDI laser to approximately 400 μm allows the entire sample to be queried simultaneously. This reduces the “sweet spot” problem often encountered when using the dried droplet method of sample preparation. Together, these factors greatly increase the sample throughput of the overall platform.

The fractionation and target plate deposition system employed in the present invention provide flexibility in the number and position of the collected samples. In one embodiment, approximately 150 nL volume aqueous droplets were precisely arrayed on a three by five square inch stainless steel plate in a 6144 microtiter array format, with each spot clearly distinguished from its nearest neighbors. The matrix can also automatically be applied using the deposition system, either before, during, or after the chromatographic process.

Mass Spectrometry of Samples

A proteomics approach based on MALDI or other LDI-type ionization procedures possess significant advantages compared to the current predominant approach of on-line coupling of separations to the mass spectrometer through electrospray ionization (ESI). For example, the samples collected and used in an LDI-based analysis platform provide a “permanent record” of the multidimensional separation by depositing the effluents of the final separation columns directly onto MALDI target plates. Decoupling the separation step from the mass spectrometer in this manner allows the chromatography to be performed free of any artificially-imposed restrictions, while allowing the mass spectrometer can operate at maximum throughput. The resulting plates can also be reanalyzed as required without the need to repeat the separation step, thus decreasing sample requirements while simultaneously greatly increasing the overall throughput of the system.

MALDI methods have recently been demonstrated on mass analyzers that are suitable for high-throughput protein identification using tandem mass spectrometry, including quadrupole ion trap, quadrupole time-of-flight, time-of-flight/time-of-flight (TOF/TOF), and Fourier transform ion cyclotron resonance. Although each system has its own operational advantages, the choice of mass analyzer to be employed in a proteomics platform must ultimately be based on which one possesses the best compromise of sensitivity, dynamic range, resolution, mass accuracy, and level of automation required for the successful analysis of complex protein mixtures.

The methods of the present invention include ionizing sample components and obtaining masses using a mass spectrometer that provides a mass accuracy of 5 ppm or better (e.g., a high mass accuracy mass spectrometer, preferably, a FT-ICR mass spectrometer). Procedures for generating MS data are well described in the art. As noted above, some embodiments of the present invention employ a modified 7 T Bruker Apex™ II FT-ICR equipped with a intermediate pressure MALDI source and a N₂ laser. Recalibration and data reduction are performed automatically, for example, using THRASH (Horn et al. (2000) J. Am. Soc. Mass Spectrom. 11:320). The resulting masses are assigned to polypeptide sequences using a matching algorithm such as PAWS (Proteometrics, New York, N.Y.).

Any matrix suitable for MALDI can be used in the present invention (see, for example, Principles of Instrumental Analysis, 5th Edition (eds. Skoog, Holler & Nieman, Harcourt Brace and Company, Philadelphia Pa., 1998) and Mass Spectrometry for Biotechnology by G. Siuzdak (Academic Press, San Diego, 1996). Exemplary matrices include, but are not limited to, α-cyano-4-hydroxycinnamic acid, sinapic acid, 2-(4-hydroxyphenylazo) benzoic acid, succinic acid, 2,6-dihydroxyacetophenone, ferulic acid, caffeic acid, glycerol, 4-nitroaniline, 2,4,6-trihydroxyacetophenone, 3-hydroxypicolinic acid, anthranilic acid, nicotinic acid, salicylamide, trans-3-indoleacrylic acid, dithranol, 2,5-dihydroxybenzoic acid, 3,5-dihydroxybenzoic acid, isovanillin, 3-aminoquinoline, T-2-(3-(4-t-butyl-phenyl)-2-methyl-2-propenylidene)malanonitrile, and 1-isoquinolinol. The matrix can be composed of one or more of these components, and/or a polymer, oligomer, and/or self-assembled monomer of one or more of these matrix components. As understood by one of skill in the art, the matrix chosen for use in the methods of the present invention will depend in part upon the analyte of interest. In some embodiments of the present invention, the matrix employed is a hydrophobic matrix; in other embodiments, a hydrophilic matrix is used.

Optionally, the ionizing and mass obtaining steps further comprise a standardization procedure. For example, the collection of the mass spectral data optionally further comprises providing one or more standards for comparison to the mass of the peak of interest, ionizing the one or more standards separately from the sample, thereby providing ionized standards, and mixing the ionized standards with an ionized sample in a gas phase. Preferred methods for performing internal calibrations on MS samples can be found, for example, in U.S. application Ser. No. ______ [Attorney Docket No. 36-003010US] and PCT application ______ [36-003010PC] co-filed herewith.

Calculation of Theoretical Mass

The sample molecular masses as determined by MS are compared to theoretical molecular masses for a plurality of in silico polypeptides or proteins during the identification process. The plurality of in silico peptides or proteins can be obtained from any of a number of sources. Optionally, the information database employed can provide either the amino acid sequences, or the nucleic acid sequences encoding the plurality of polypeptides. Thus, either amino acid or nucleic acid sequence listing can be used to generate the plurality of in silico peptides.

Sequences can be obtained from any of a number of private or commercial databases. In many embodiments of the present invention, the in silico polypeptides represent a proteomic database, such as the “Proteome BioKnowledge Library” available from Incyte Genomics, Inc. (see, for example, www.incyte.com/sequence/proteome). Other sources include, but are not limited to, the GenBank® databases (available from the National Center for Biotechnology Information, www.ncbi.nlm.nih.gov), the NCBI EST sequence database, the EMBL Nucleotide Sequence Database; various nucleotide and protein databases provided by the European Bioinformatics Institute (www.ebi.ac.uk), and proprietary databases available from companies such as Incyte (Palo Alto, Calif.) and Celera (Rockville, Md.). In some embodiments, the methods employ in silico polypeptides derived from amino acid sequences encoded by one or more members of members of a genomic nucleic acid library, or an EST library. Furthermore, the databases employed may be specific for a particular species (e.g., human, mouse, rat, Drosophila, yeast, bacterium, etc.) or a specific type of encoded molecule (e.g., pharmaceutically-relevant gene families, protein super families, phylogenetically related sequences, and the like.

For embodiments in which the sample proteins have been cleaved by a proteolytic agent, the calculation of theoretical masses also includes examining the amino acid sequences and identifying one or more predicted cleavage sites for the selected proteolytic reagent. This information can be used to provide sequences of the in silico proteolytic peptides that would be obtained by cleavage of the protein at one or more of the predicted cleavage sites. Since proteolysis of the sample peptides typically generates combinations of all possible cleavage products (e.g., not every cleavage site is accessed during proteolysis), the in silico proteolysis products optionally reflect the incomplete nature of the proteolysis reaction. In the methods of the present invention, the in silico proteolytic peptides optionally comprise peptides having up to three missed enzymatic cleavage sites. Furthermore, the in silico peptide fragments can be selected to range in molecular mass, for example, from 500 Da to 10,000 Da, or from 1000 Da to 6000 Da, or other selected size ranges.

The methods of the present invention also take into account the incomplete nature of chemical and biochemical reactions. For example, preparation of the list of computer-generated proteolytic peptide fragments allows for inclusion of polypeptides having 1, 2, 3, or more missed cleavage sites (e.g. incomplete digestion). As a means of reducing the list of theoretical peptides thus generated, the product in silico peptides can also be selected by size (molecular mass) prior to inclusion in the in silico peptide database. For example, the in silico peptides can range in molecular from about 500 Da to about 10,000 Da. In an alternative embodiment, the in silico proteolytic peptides range in molecular mass from 1000 Da to 6000 Da.

Further Analytical Steps

Occasionally, one or more fractions of the sample will contain a polypeptide or peptide fragment for which no unambiguous match was observed among the in silico polypeptides. For these situations, the methods of the present invention optionally comprise subjecting that fraction to further analysis to identify the proteolytic peptide that is present in the fraction. The further analysis can be performed by an comparing the MS data generated for the fragment with theoretical masses generated for an alternate database of protein sequences. Alternatively, the fraction can be further analyzed by an alternative analytical methods, such as tandem MS.

The methods of the present invention also include the optional step of generating one or more additional databases of proteolytic peptide sequences for comparison purposes. The member proteolytic peptides optionally i) are derived in silico from the amino acid sequences in either the identified protein database or the theoretical protein database (e.g., the universe of proteins) by predicted action of one or more additional proteolytic reagents upon members of the database; ii) encompass peptide sequences having 1, 2, 3 or more missed enzymatic cleavage sites; and iii) fall within a desired size range (e.g., between 500 Da and 10,000 Da, or 1000 Da and 6000 Da, or 1000 Da and 4000 Da).

Systems for Protein Identification

The present invention also provides systems for identifying a plurality of member proteins in a sample. Optionally, the plurality of member proteins are treated with at least a first proteolytic reagent, thereby generating proteolytic peptides for MS analysis. The systems comprise a) an ionization source and a mass spectrometer that provides a mass accuracy of 5 ppm or better; b) an interface for receiving mass spectral data from the mass spectrometer; c) a database of theoretical molecular masses of protein sequences or proteolytic peptides; and d) a computer or computer-readable medium in communication with the interface and the database of theoretical molecular masses. The computer (or computer-readable medium) of the system further comprises instructions for determining the mass of two or more sample polypeptides from the mass spectral data mass peaks, and comparing the determined mass to members of the database of theoretical molecular masses.

As noted previously, a preferred mass spectrometer for use in the systems of the present invention is an FT-ICR mass spectrometer. The ionization source is preferably a MALDI source and can include e.g., a vacuum source, an intermediate pressure source, or an atmospheric pressure source.

Optionally, the interface for receiving the MS data and the computer (or computer-readable medium) comprise a single unit for collection and analysis of the data. In some embodiments of the device, the interface further comprises software for both generating and processing of the mass spectral data by the mass spectrometer.

The systems of the present invention can also comprise a fractionation system (e.g., a liquid chromatography system), optionally coupled fluidically to an automatable sample collection system. In a preferred embodiment, the fractionation system is a reverse phase μHPLC system, providing either a single column or an array of columns. Typically, the sample collection system includes an eluent collection plate that is configured for use in the mass spectrometer of the system. One embodiment of the eluent collection plate comprises a hydrophobic surface and one or more hydrophilic regions, commonly referred to as a hydrophobic/hydrophilic plate.

Optionally, in an integrated fractionation/data collection system embodiment of the present invention, the system comprises a sample source and a source of one or more proteolytic reagents, wherein the sample source and the source of proteolytic reagents are fluidically coupled to one another through a mixing region, and wherein the mixing region is fluidically coupled to the liquid chromatography system. In some embodiments, sample and reagent sources, the mixing regions, and optionally the fractionation system, comprise one or more microfluidic systems. See, for example, U.S. Pat. No. 6,235,471 to Knapp et al. (Caliper Technologies, Corp., Mountain View, Calif.; www.calipertech.com) and lab stations and equipment available from Gyros US, Inc. (Monmouth Junction, N.J.; www.gyros.com).

Typically, the MS data generated by the systems of the present invention comprise mass peaks obtained from a sample that was contacted with at least a first derivatizing agent that specifically labels a selected amino acid or functional moiety when the selected amino acid or functional moiety is present in a protein in the sample. The derivatizing component of the newly-formed complex shifts the mass of the peptide a set amount, depending upon which isotopic form is bound. The system optionally comprises a mechanism for accommodating the increased mass of the labeled sample peptide as compared to an in silico peptide, by providing either a) instructions for subtracting the molecular mass of the derivatizing agent (multiplied by the number of occurrences of the selected amino acid in the proteolytic peptide) from the observed molecular mass for the proteolytic peptide, or b) instructions for adjusting the theoretical molecular mass calculated for the in silico peptide by adding the appropriate molecular mass of the derivatizing agent(s) to the in silico peptide prior to comparison with the observed molecular mass for the proteolytic peptide. Optionally, the instructions also accommodate incomplete proteolytic action by providing in silico proteolytic peptides having up to three missed enzymatic cleavage sites, and optionally ranging in size from 500 Da to 10,000 Da, or from 1000 Da to 6000 Da.

The systems of the present invention can also include, but are not limited to, one or more additional databases of in silico polypeptides (optionally, proteolytic peptides). The member in silico proteolytic peptides of the additional databases optionally i) are derived in silico from a database of protein sequences by action of one or more additional proteolytic enzyme upon members of the database. Furthermore, the peptides can be selected for inclusion in the database of in silico proteolytic peptides based upon extent of completion of the cleavage reaction (e.g., including peptide sequences having up to three missed enzymatic cleavage sites) and/or size (e.g. only those peptides ranging in size between 1000 Da and 6000 Da.)

In some embodiments, the system is used to generate and examine mass spectral data obtained from a sample that was contacted with at least a first derivatizing agent that specifically labels a selected amino acid or functional moiety when the selected amino acid or functional moiety is present in a protein in the sample. Typically in this embodiment, the system also comprises instructions for adjusting the molecular mass determined for a proteolytic peptide by adjusting (e.g., subtracting from) the observed molecular mass of the proteolytic peptide by the molecular mass of the derivatizing agent multiplied by the number of occurrences of the selected amino acid in the proteolytic peptide. Alternatively, the systems of the present invention comprise one or more of a) instructions for generating a subset of in silico proteolytic peptides that comprise a selected amino acid to which the derivatizing agent can attach; b) instructions for calculating molecular masses for the subset of in silico proteolytic peptides having an attached derivatizing agent; and c) instructions for comparing the molecular masses for the derivatized in silico proteolytic peptides to the mass peaks for the labeled sample polypeptides.

As a further means of data complexity reduction, the system optionally includes a) instructions for generating a subset of in silico proteolytic peptides that comprise a selected amino acid to which the derivatizing agent can attach; b) instructions for calculating molecular masses for the subset of in silico proteolytic peptides having an attached derivatizing agent; and c) instructions for comparing the molecular masses for the derivatized in silico proteolytic peptides to the mass peaks for the sample proteolytic peptides. In this manner, only the in silico peptides having the labeled amino acid are scanned for matches to the experimental mass data.

Optionally, the systems of the present invention further comprise one or more additional databases of in silico proteolytic peptides, wherein the member in silico proteolytic peptides of the additional databases are derived in silico by action of one or more additional proteolytic enzyme. Thus, the additional databases reflect alternative proteolytic “profiles” of the first sequence database, which, when combined with an alternative proteolytic cleaving of the sample proteins, increases the probability that a selected sample protein can be identified.

As a means of data complexity reduction, the systems of the present invention optionally include instructions for calculating theoretical molecular masses for any additional in silico proteolytic peptides derived from a previously-identified protein (e.g., as identified in the comparison of the mass obtained for the first proteolytic peptide to the theoretical molecular masses), and disregarding mass spectral data collected for additional sample peptides if the mass spectral data for the additional peptide matches that which would be obtained for one or more of the additional in silico proteolytic peptides from the previously identified protein. These instructions can be performed simultaneously (e.g., the computer or computer readable medium simultaneously compares two or more sample masses to the theoretical molecular masses for the in silico proteolytic peptides) or sequentially (e.g., comparison of any additional sample mass spectral data to the theoretical mass database is performed after identification of the first protein). An exemplary program for performing the comparison and identification (on a single MS peak/peptide) is the Mascot Daemon program from Matrix Science Ltd. (London, Great Britain). Additional software for data comparison and identification can be generated by one of skill using standard software language.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### MS Data for a Portion of a Yeast Proteome

One advantage of the methods and systems of the present invention over protocols in the prior art is the capacity for analysis of complex populations of proteins containing thousands of elements. Simplification of the mixture of peptides is not required, unlike as is done in the ICAT strategy where only at most a few peptides per protein will be present in the mixture analyzed by the mass spectrometer. Thus, tens to hundreds of peptides from the same protein can be characterized by the mass spectrometer.

FIG. 2 provides a representation of the reduced data in three-dimensional space spanned by mass, fraction number (called “spot” in the figure), and signal-to-noise ratio for a soluble yeast protein extract. The extract was prepared, reduced, alkylated, and digested with trypsin; 5 μg of this digest was separated on a 300 μm i.d. reversed-phase μHPLC column run at 3 μl/min, and 10 s fractions of the effluent were codeposited with matrix onto a MALDI plate. Over 11,000 unique masses were found in this data set, with a considerable number of spectra that exhibiting over 200 masses. The typical dynamic range observed in these spectra was 500 and in quite a few cases the dynamic range was over 1000. In additional experiments an identical sample was first fractionated by strong cation-exchange (SCX) before μHPLC. The sample was eluted in four salt steps from the SCX column, each of which was simultaneously separated and deposited with matrix onto a 1536 format MALDI target plate. Analyses of these samples detected a similar number of peptides in each SCX fraction as seen previously for a single RP-μHPLC separated sample. This demonstrates the increase in overall peak capacity of the system if further up-front separation steps are employed.

### Example 2

### Database Sequence Coverage Experiments

To assess the utility of lysine and acidic amino acid-specific accurate mass tags, computer simulations were performed on two different non-redundant databases: a first database derived from yeast (from NCBI, 6298 entries) and a second database based upon human sequences (from European Bioinformatics Institute, 32513 entries). All possible proteolytic peptides in the mass range 1000-4000 Da were determined by in silico digestion of each protein entry in the database using five different proteases (ArgC, AspN, GluC, LysC, trypsin). A maximum of 2 missed cleavages were allowed per peptide sequence. For each peptide, it was determined whether or not another peptide exists within a given ppm error (1, 5, 10, and 50 ppm) and, if so, whether or not they contain the same number of lysines and/or acidic amino acids. The data is summarized in three different manners, reflecting: 1) the effect of the mass accuracy on the number of proteins identified, 2) the effect of the knowledge of the number of a given amino acid type on the ability to identify proteins by the accurate mass of a single peptide, and 3) the effect of using data from more than one proteolytic digest on increasing the coverage of the proteome.

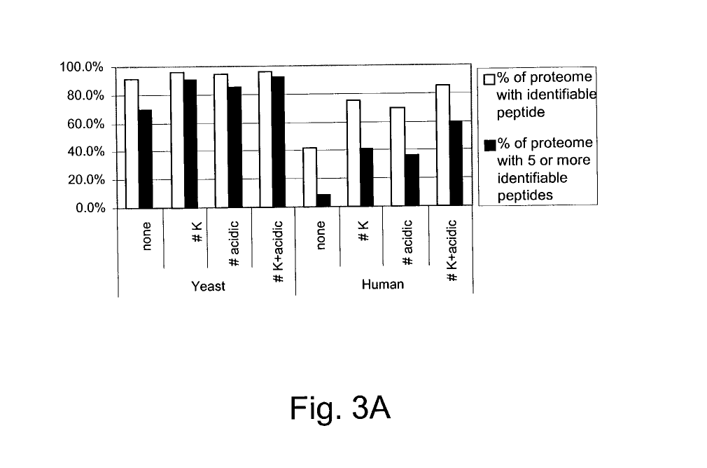

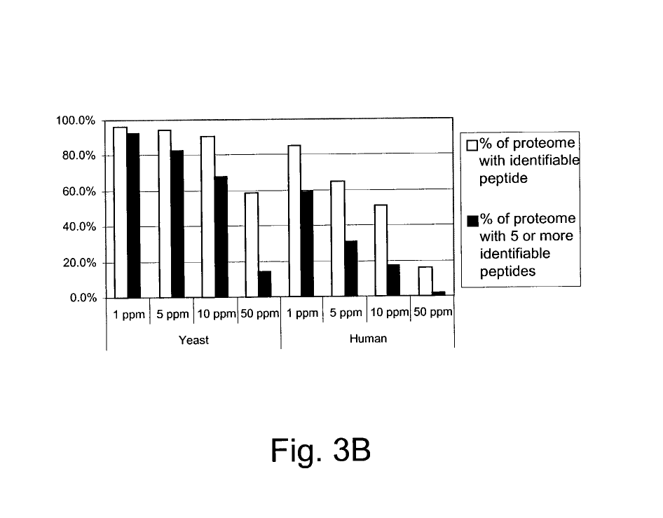

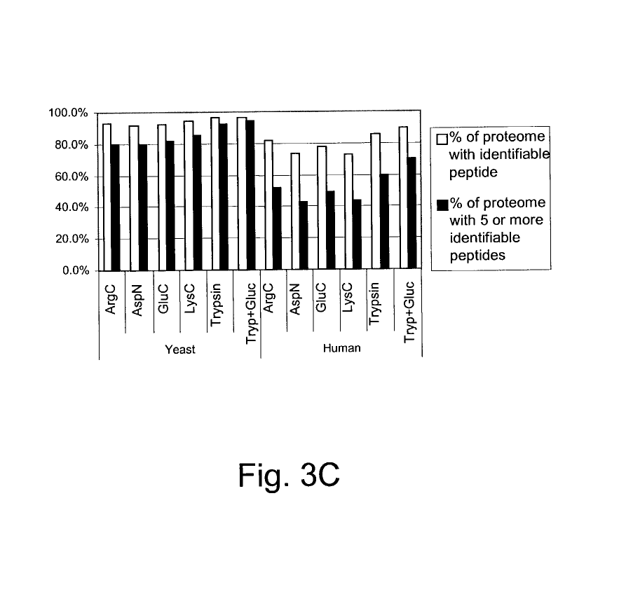

The percentage of proteins in the database that can be identified given a 1 ppm mass accuracy, and optionally using information regarding the number of lysines and/or acidic amino acids present in the protein, is provided in FIG. 3A. The graph illustrates that it is more advantageous to know two (or more) sequence-specific factors, such as both the number of lysines and the number of acidic amino acids in a peptide, especially for the human proteome. In addition, the second (shaded) set of data bars in FIG. 3A represent the percentage of proteins that contain 5 or more uniquely identifiable peptides (e.g., proteins for which there is a far greater likelihood of the identification). The complete digest of a protein generally results in 100-150% sequence coverage, but the simulations include all peptides up to 2 missed cleavages, corresponding to 600% sequence coverage. Thus, proteins that generate at least 5 peptides (including incomplete digestion fragments) should have a significant chance (>50%) of being detected and identified by the provided methods.

Given the knowledge of both the number of lysines and acidic amino acids in a peptide, FIG. 3B demonstrates the effect of mass accuracy on the number/percentage of proteins that may be identified using the accurate mass strategy. Each of the provided mass accuracy data sets (1 ppm, 5 ppm, 10 ppm and 50 ppm) represents the best mass accuracy that can typically be obtained by a type of instrument: a 50 ppm mass accuracy for MALDI-TOF, a 10 ppm mass accuracy by typical TOF mass accuracy, a 5 ppm mass accuracy by orthogonal extraction TOF at its unlikely best, and 1 ppm mass accuracy can be obtained by FT-ICR. The data indicates (especially for the human proteome database) that 1 ppm mass accuracy gives significantly more coverage of the proteome sequence than even 5 ppm, thus indicating that the use of FT-ICR in this application is a preferred method of generating mass data.

FIG. 3C depicts the percentage of identifiable proteins in the yeast or human proteome databases after in silico protease treatment. The graph demonstrates that trypsin provides greater coverage of the proteome sequence than the other proteolytic enzymes examined. This result is most likely due to the larger number of peptides in the selected mass range (between 1000 and 4000 Da) that are created by trypsin as compared to the other proteases. Combination of the GluC and trypsin digests suggests that the information generated via examination of the proteolytic digests is complementary. The combination increased/improved the sequence coverage of the human proteome with 5 or more peptides from 60% with trypsin to 70% for both GluC and trypsin, which is a gain in the ability to identify over 3000 more proteins. However, such a step is unnecessary with the yeast proteome data set, as only 2% more sequence coverage is obtained; identification of these proteins by tandem MS would probably take less time than a complete separation and MS of the second proteolytic digest. The data indicate that an accurate mass approach to protein identification incorporating the knowledge of the number of one or more specific amino acid types is feasible for proteomes as large as the human, and is quite straightforward for proteomes the size of yeast. Since the majority of proteins can be identified in this manner for both proteomes, the analysis time for proteome profiling will decrease significantly due the greatly reduced number of tandem MS experiment that will be required.

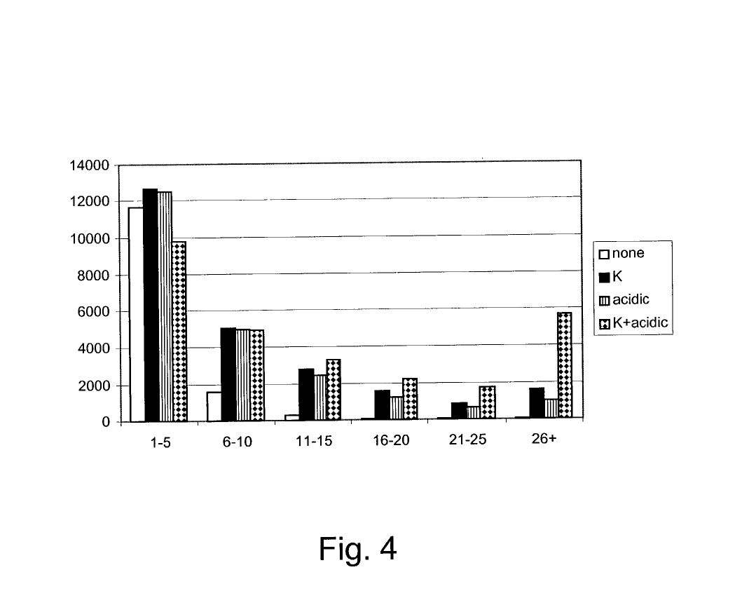

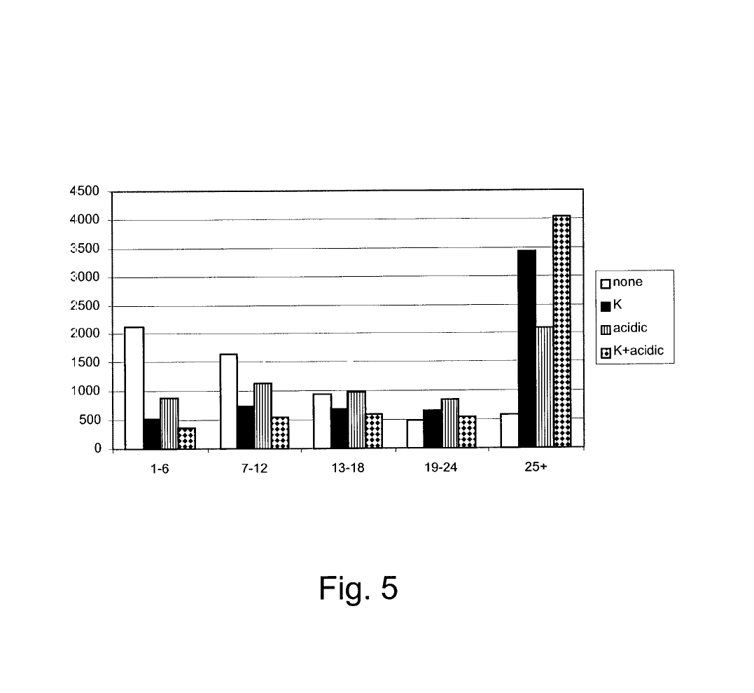

FIG. 4 and FIG. 5 depict the effect that derivatization (via lysine and/or acidic amino acid-specific accurate mass tags) has on the number of identifiable peptides per protein in either the yeast proteome or the human proteome, respectively. Data is based upon data sets generated at 1 ppm mass accuracy.

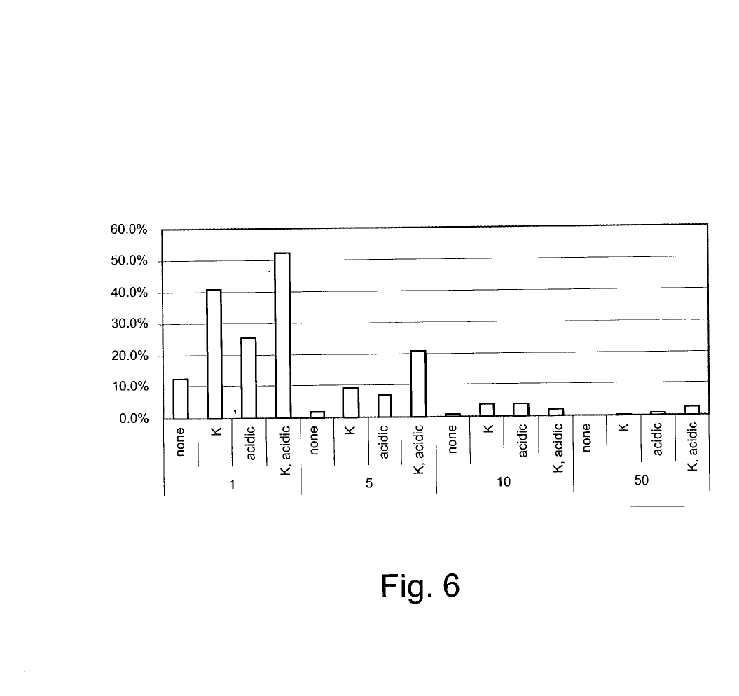

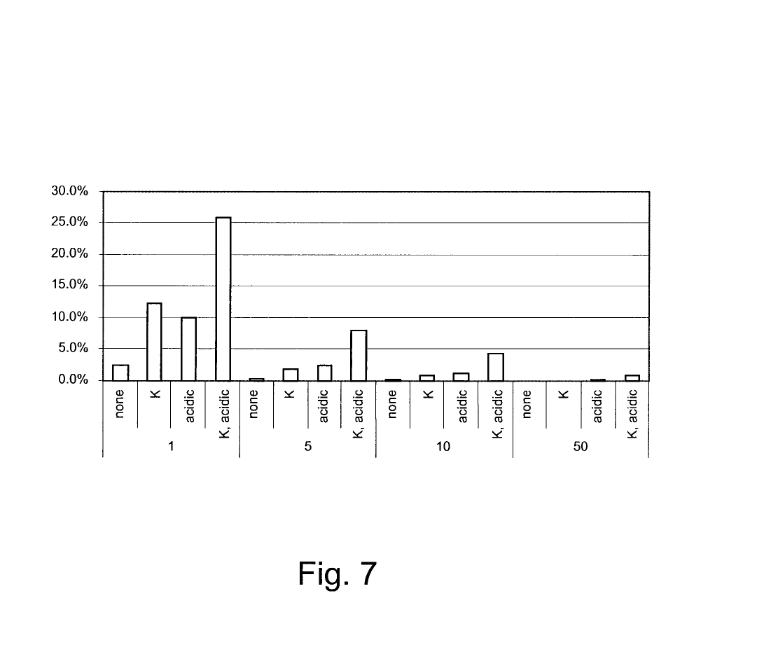

FIG. 6 and FIG. 7 demonstrate the effect of mass accuracy (1 ppm, 5 ppm, 10 ppm or 50 ppm) and derivatization strategy (lysine and/or acidic amino acid-specific accurate mass tags) on data generation for tryptic digests of yeast and human proteins, respectively.

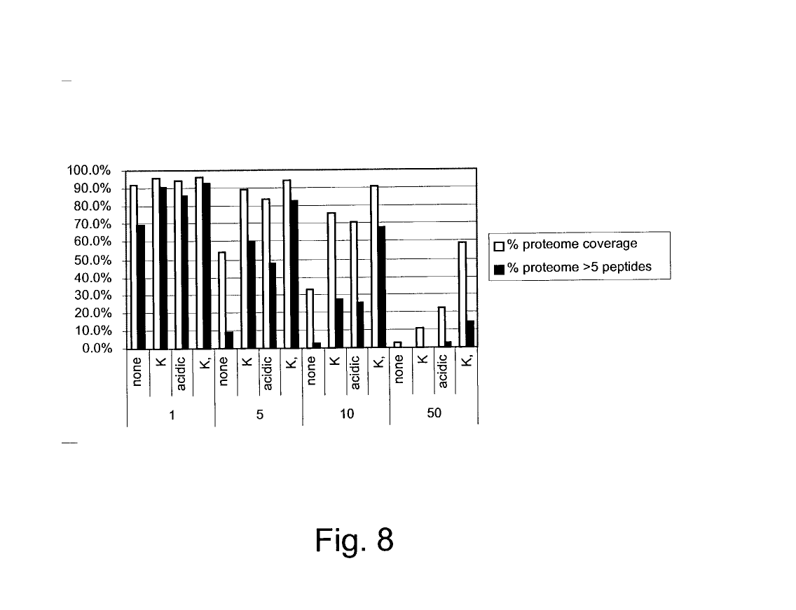

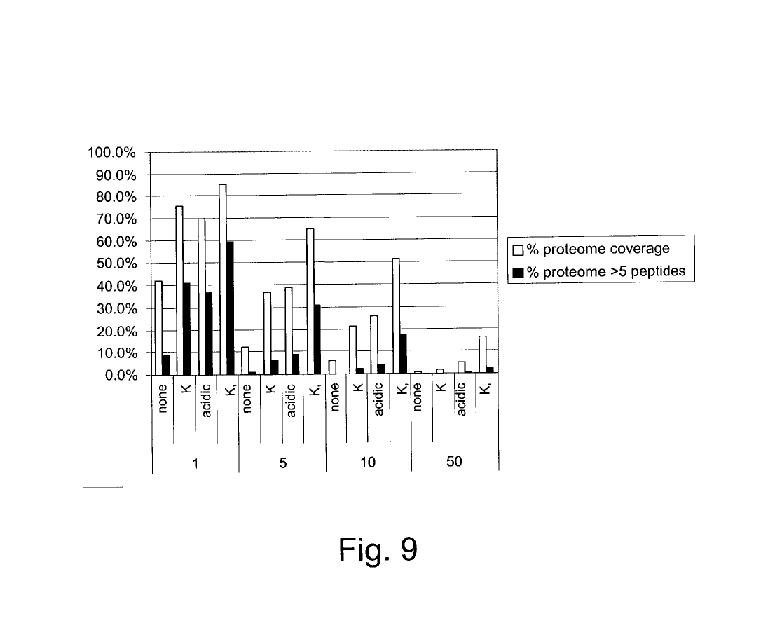

FIG. 8 and FIG. 9 show the effect of mass accuracy and derivatization strategy on yeast and human proteome coverage, respectively.

### Example 3

### Assignment of PTM-Peptides from Unidentified Masses

Using the accurate mass and CRAMP techniques described herein, and possibly tandem MS if necessary for assignment confirmation, it is expected that all possible proteins present in the sample have been identified. Thus, any remaining unassigned masses are assumed to contain one or more modifications of a proteolytic peptide from one of the already identified proteins. Given that the exact masses for many modifications are already known, all combinations of masses of one or more of the modifications are subtracted from the measured mass (with 1 ppm accuracy) and used with the potential knowledge of the number of one or more amino acids in the peptide, expression ratio, and any other distinguishing information. These sets of masses are compared to the unmodified peptide sequences from an in silico digest of the complete set of identified proteins and any match indicates the possible assignment of that peptide with the post-translational modifications. If there is more than one match, the peptide may be subjected to tandem MS, which will likely be able to distinguish between the possibilities.

As noted previously, an interesting feature of mass data collected for peptides having post-translational modifications (PTM-peptides) is the “mass defect” effect. This information can be used to determine whether unassigned peaks in the mass spectral data can be accounted for by the presence of a post-translational modification. To assess the effect of the mass defect of a phosphate group on the ability to uniquely identify phosphopeptides, computer simulations were performed on the a second human proteome database (European Bioinformatics Institute, having 36493 sequences).

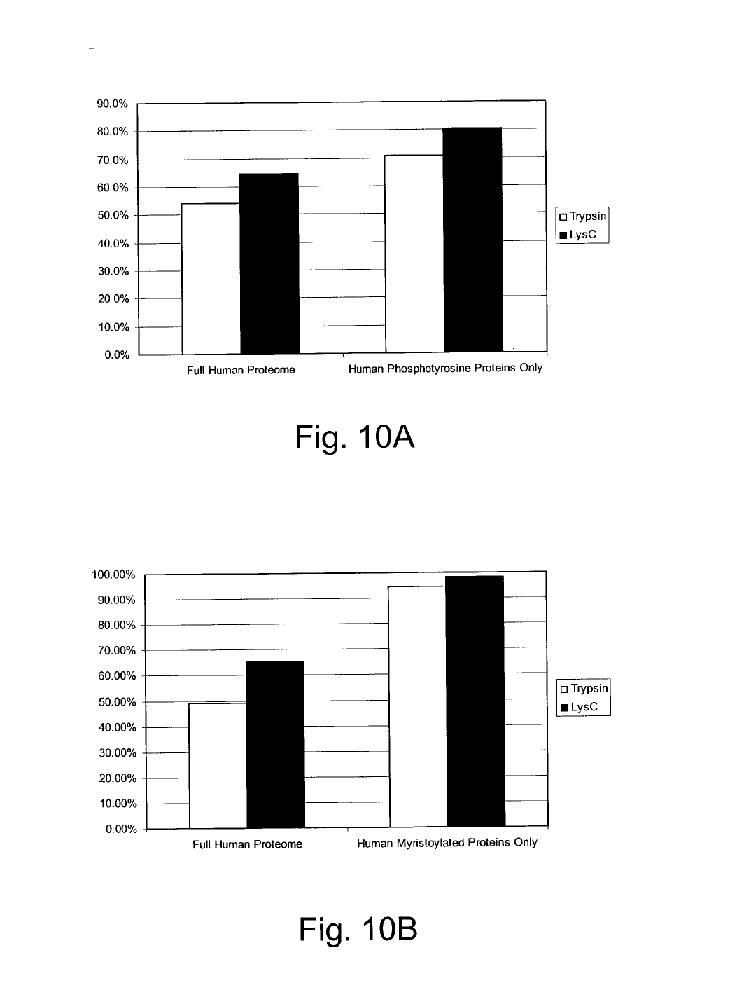

Tyrosine phosphorylation is typically found on peptides having one of two sequence motifs: [(R or K)XX(D or E)XXXY] or [(R or K)XXX(D or E)XXY], where X represents any amino acid (as obtained from PROSITE at us.expasy.org/prosite). All proteins in the database that contained at least one of the sequence motifs were assumed to have an attached phosphate group on the tyrosine. A second, simplified database that only contains theses proteins (6984 total sequences) was generated. All possible proteolytic peptides in the mass range 1000-4000 Da were calculated by in silico digestion of both the complete proteome database and the motif-containing second sequence database, using two different proteases (trypsin, LysC), and allowing for a maximum of 2 missed cleavages per peptide. For each possible phosphopeptide, it was determined whether or not there was another peptide whose mass was within 1 ppm that contained the same number of lysines and acidic amino acids. FIG. 10A shows the percentages of phosphopeptides that are uniquely identifiable given 1 ppm mass accuracy and lysine and acidic amino acid specificity. For trypsin, over half of the phosphopeptides show unique mass and amino acid information, and thus these peptides will not be assigned by CRAMP to another protein, while with LysC almost 65% of the phosphopeptides are identifiable. When only considering the phosphotyrosine containing proteins (which can be enriched experimentally by phosphotyrosine antibodies), these percentages go up to 70.9% for a trypsin digest and 80.3% for LysC.

A similar test was performed on the myristoylation post-translational modification (FIG. 10B). A myristoyl group was added to all proteins from the human EBI database that contained an N-terminal glycine and the full database and the simplified database containing only the modified proteins (1315 total) were created and in silico digested as above. It was found that again for trypsin, about half of the modified peptides were uniquely identifiable (49.1%) and 65% of LysC peptides are identifiable. Due to the fewer number of modified proteins, the simplified database showed a much larger number of identifiable proteins: 94.4% for trypsin and 98.2% for lysine.

### Example 4

### MALDI Experimental Setup

An exemplary MS experiment is described. A 384 or 1536-micro-titer format target plate containing deposited analytes is mounted onto linearly encoded high precision x- and y-stages in a custom-built intermediate pressure MALDI source. Following UV laser irradiation, the generated ions are collisionally cooled by the surrounding nitrogen buffer gas (pressure of 40 mTorr) and guided by a cooling quadrupole to the entrance of a selection quadrupole, through which they are passed into a hexapole ion guide for transient storage. The selection quadrupole can be operated in integral or mass selective mode, allowing the isolation of a narrow mass range before ion accumulation. Internal calibration, which is required to ensure the high mass accuracy inherent in FT-ICR MS, is achieved by employing a novel gas phase mixing scheme (see U.S. application Ser. No. ______ [Attorney Docket No 36-003010US] and PCT application ______ [Attorney Docket No. 36-003010PC] co-filed herewith). Specifically, after sample irradiation and storage of the resulting ions in the hexapole, the stage quickly moves to a strip containing peptide calibrants imbedded in a MALDI matrix located on the edge of the plate. Calibrant ions are then generated and mixed with the sample ions in the hexapole, and the entire packet is transferred into the mass analyzer. Software has been written to both automate the acquisition of mass spectra without user intervention as well as deconvolute the resulting isotopic clusters (Horn, supra). The total time required for the acquisition of a typical mass spectrum is roughly 7 to 10 seconds, enabling internally calibrated mass spectra for 384 samples to be acquired in less than 1 hr. Similarly, automated tandem MS can be performed in the analyzer cell by sustained off-radiance irradiation collisionally activated dissociation (SORI-CAD) or by infrared multi-photon dissociation (IRMPD).

### Example 5

### Resolution Effects in a Differential Display Experiment

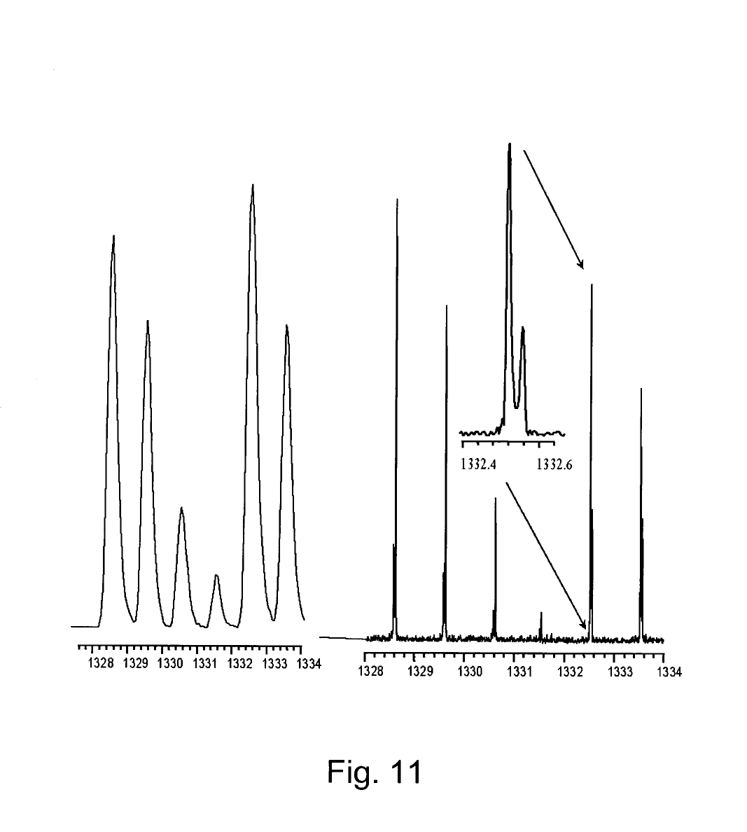

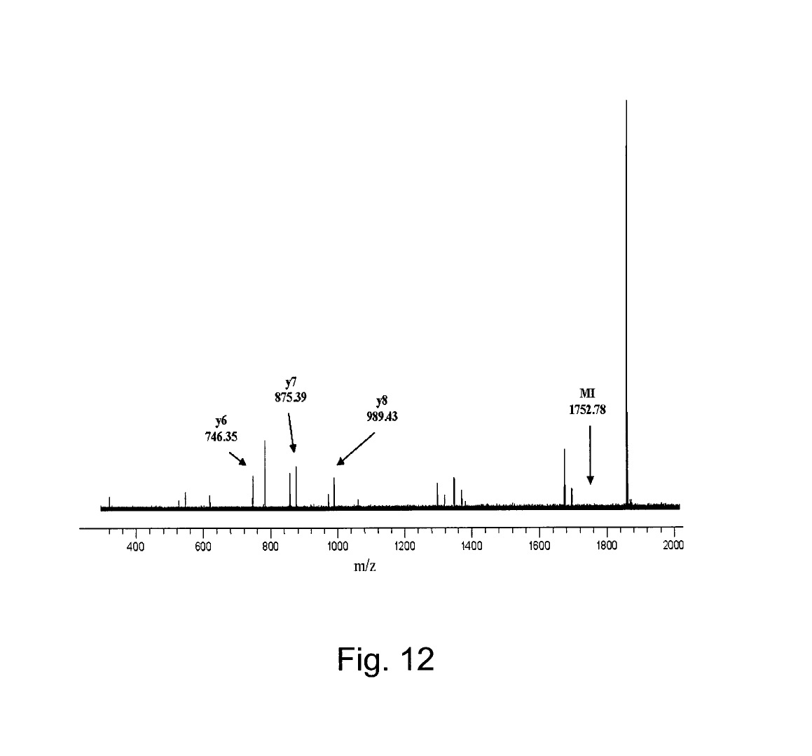

FIG. 11 demonstrates the utility of high resolution measurements in a simulated differential display experiment (Moseley (2001) Trends Biotechnol 19:S10-S16. Two peptides differing in mass by 40 mDa were labeled separately with a 1:3 mixture of the N-Hydroxysuccinimide esters of nicotinic acid: d₄-nicotinic acid for the lower mass peptide or 3:1 for the larger mass species. Equal amounts of each labeled peptide were combined and a mass spectrum of the resulting mixture was obtained on both a MALDI-TOF and our MALDI FT-ICR. The spectrum from the MALDI-TOF shows what appears to be a single peptide labeled in a 1:1 ratio, whereas the high resolution of the FT-ICR mass spectrum clearly shows the presence of the two differentially-labeled isotopic clusters. A resolution of at least 33,000 is required according to the full-width half maximum (FWHM) criterion in order to resolve the signals of the two peptides. Such high resolution measurements are only feasible using FT-ICR MS. For extremely complex mixtures containing hundreds of thousands of peptides, lower resolution measurements may result in the loss or misinterpretation of data as demonstrated by the MALDI-TOF spectrum.

### Example 6

### Protein Identification of a Shikimate 5-dehydrogenase Tryptic Digest

The high mass measurement accuracy afforded by FT-ICR is also highly advantageous for protein identification. Table 1 shows the database search results for an internally-calibrated peptide map of a shikimate 5-dehydrogenase (Thermotoga maritima) tryptic digest. The root-mean-squared mass accuracy of 3 ppm for assigned peptides spanning a range of 1700 m/z (69% sequence coverage) resulted in the unambiguous identification of shikimate 5-dehydrogenase from the NCBI non-redundant database using the Mascot protein identification software, which returned a score of 259. Since a score of 45 for this search indicates 95% confidence in the protein identification and the returned Mascot score is proportional to the negative of the logarithm of the probability (Perkins et al. (1999) Electrophoresis 20:3551:3567), there is a ˜10⁻²⁵ percent chance that this identification is incorrect. Furthermore, the next most probable match is assigned a score of only 19, which is significantly below the confidence threshold. This spectrum was acquired as part of an automated MS run of tryptic digests of 96 protein samples. The entire process including data acquisition with internal calibration, data reduction, and protein identification was completed in less than two hours total. Of these 96 samples, 91 were unambiguously identified in the NCBI non-redundant database, most with Mascot scores well above 100, while the remaining five samples could not be identified due to insufficient protein concentration.

TABLE 1

List of molecular masses and peptide fragments

| | | | | | | | | |
|---|---|---|---|---|---|---|---|---|
| | | | | | | ppm | | |
| Start | End | Observed | Mr(expt) | Mr(calc) | Delta | Error | MCS | Sequence |

| | | | | | | | | |
|---|---|---|---|---|---|---|---|---|
| 18 | 24 | 975.4764 | 975.4764 | 975.4702 | 0.0062 | 6.4 | 0 | LYNEYFK |
| 18 | 25 | 1131.5742 | 1131.5742 | 1131.5713 | 0.0029 | 2.6 | 1 | LYNEYFKR |
| 26 | 47 | 2509.1064 | 2509.1064 | 2509.0889 | 0.0175 | 7.0 | 0 | AGMNHSYGMEEIPPE |
| | | | | | | | | SFDTEIR |
| 26 | 48 | 2665.2114 | 2665.2114 | 2665.19 | 0.0214 | 8.0 | 1 | AGMNHSYGMEEIPPE |
| | | | | | | | | SFDTEIRR |
| 48 | 63 | 1901.97 | 1901.97 | 1901.9635 | 0.0065 | 3.4 | 1 | RILEEYDGFNATIPHK |
| 49 | 63 | 1745.869 | 1745.869 | 1745.8624 | 0.0066 | 3.8 | 0 | ILEEYDGFNATIPHK |
| 49 | 65 | 2031.0105 | 2031.0105 | 2031.0061 | 0.0044 | 2.2 | 1 | ILEEYDGFNATIPHKE |
| | | | | | | | | R |
| 69 | 78 | 1192.5413 | 1192.5413 | 1192.536 | 0.0053 | 4.4 | 0 | YVEPSEDAQR |
| 90 | 100 | 1236.6194 | 1236.6194 | 1236.6139 | 0.0055 | 4.4 | 0 | GYNTDWVGVVK |
| 101 | 121 | 2022.1064 | 2022.1064 | 2022.1109 | −0.0045 | −2.2 | 1 | SLEGVEVKEPVVVVG |
| | | | | | | | | AGGAAR |
| 109 | 121 | 1180.6617 | 1180.6617 | 1180.6564 | 0.0053 | 4.5 | 0 | EPVVVVGAGGAAR |
| 154 | 166 | 1532.8445 | 1532.8445 | 1532.845 | −0.0005 | −0.3 | 1 | IFSLDQLDEVVKK |
| 169 | 191 | 2453.2175 | 2453.2175 | 2453.1995 | 0.018 | 7.3 | 1 | SLFNTTSVGMKGEEL |
| | | | | | | | | PVSDDSLK |
| 192 | 209 | 2097.1427 | 2097.1427 | 2097.1397 | 0.003 | 1.4 | 0 | NLSLVYDVIYFDTPL |
| | | | | | | | | VVK |
| 221 | 234 | 1720.8057 | 1720.8057 | 1720.7953 | 0.0104 | 6.0 | 0 | GNLMFYYQAMENLK |
| 235 | 245 | 1397.6877 | 1397.6877 | 1397.6867 | 0.001 | 0.7 | 0 | IWGIYDEEVFK |
| 235 | 253 | 2299.1814 | 2299.1814 | 2299.1776 | 0.0038 | 1.7 | 1 | IWGIYDEEVFKEVFG |
| | | | | | | | | EVLK |

MCS: missed cleavage sites

For comparison, the same samples were analyzed on a MALDI TOF instrument, which required several days of work and resulted in just 61 protein identifications with scores above the statistical threshold of 45. The average top score for TOF data was 63.5 versus 101.5 for FT-ICR, and the average score difference between first and second assignments was 38.8 for TOF data and 79.9 for FT-ICR data. These results clearly demonstrate the benefits of high mass accuracy and high throughput afforded by using FT-ICR MS.

### Example 7

### Identification of Unknown Proteins

High mass accuracy is also extremely powerful for tandem MS experiments. FIG. 4 shows the SORI-CAD spectrum of an unknown peptide originating from a tryptic digest of all the soluble cytosolic proteins in yeast. While only three peptide fragments were detected in this experiment, this data was sufficient to unambiguously identify glyceraldehyde 3-phosphate dehydrogenase using the Mascot protein identification software due to the high mass measurement accuracy for both the parent and fragment ions (2 ppm error). The stringent search specificities employed (10 ppm for the parent ion, 0.020 Da for fragment ions) were enough to eliminate any possibility that this could be any other tryptic peptide in the whole yeast proteome. Thus, even with limited sequence information, the high mass accuracy of FT-ICR MS allows unambiguous assignment of peptides subjected to tandem MS.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of reducing a number of peaks to further be analyzed in a mass spectrum for a sample, the method comprising:
generating a first amino acid sequence database comprising an amino acid sequence of at least one protein known to be present in the sample;
calculating a first list of theoretical masses for a first set of in silico peptides generated from one or more of the amino acid sequences in the first database; and
correlating the first list of theoretical masses with positions of the unidentified MS peaks and identifying one or more MS peaks that correspond to masses for the in silico peptides, thereby reducing the number of peaks to further be analyzed in the mass spectrum.

2. The method of claim 1, wherein all members of the first database are proteins known to be present in the sample.

3. The method of claim 1, wherein the sample comprises a plurality of proteolytic peptides generated by action of a proteolytic agent upon member proteins in the sample, and wherein calculating the first list of theoretical masses comprises generating the in silico proteolytic peptides using cleavage parameters of the proteolytic agent.

4. The method of claim 1, wherein the unidentified MS peaks are obtained using a mass spectrometer that provides a mass accuracy of 5 ppm or better.

5. The method of claim 1, wherein the unidentified MS peaks are obtained using a mass spectrometer that provides a mass accuracy of 1 ppm or better.

6. The method of claim 1, wherein generating the first database comprises providing amino acid sequences derived from protein sequencing data, nucleic acid sequencing data, tandem MS data or 2DE-MS data.

7. The method of claim 1, wherein generating the first database comprises i) selecting an unidentified MS peak and performing tandem mass spectrometry, thereby identifying a corresponding peptide sequence; and ii) determining a parent protein sequence comprising the identified corresponding peptide sequence; and wherein calculating the first list of theoretical masses comprises calculating masses for additional in silico peptides from the determined protein sequence.

8. The method of claim 7, wherein correlating the first list of theoretical masses with positions of the unidentified MS peaks further comprises identifying additional MS peaks that correspond to the theoretical masses of the additional it silico peptides and removing the additional MS peaks from a data set of unidentified MS peaks.

9. The method of claim 1, wherein generating the first database comprises:
providing a mass peak list comprising the positions of the unidentified MS peaks of the sample, wherein the MS peaks represent a plurality of proteolytic peptides generated by action of a proteolytic agent upon member proteins in the sample.
providing a second list of theoretical masses for a plurality of in silico proteolytic peptides generated from a second database of protein sequences by the in silico action of the proteolytic agent upon member sequences in the second database; and
comparing the second list with the mass peak list, thereby assigning corresponding MS peaks and identifying additional member proteins of the sample for inclusion in the first database.

10. The method of claim 9, further comprising:
regenerating the first database to include sequences for the identified additional member proteins; and
repeating the calculating, correlating and regenerating steps until no additional member proteins are identified.

11. The method of claim 9, wherein the second list comprises a first set of unique masses representing unique peptide sequences and a second set of masses representing more than one peptide sequence, and wherein comparing the second list with the mass peak list comprises comparing the first set of unique masses with the mass peak list.

12. The method of claim 11, wherein comparing the first set of unique masses with the mass peak list further comprises performing tandem MS on selected members of the plurality of proteolytic peptides, thereby confirming the identity of the additional member proteins of the sample.

13. The method of claim 9, wherein the proteolytic agent comprises a proteolytic enzyme.

14. The method of claim 13, wherein the proteolytic enzyme is selected from the group consisting of trypsin, chymotrypsin, endoprotease ArgC, aspN, gluC, and lysC.

15. The method of claim 9, wherein the proteolytic reagent comprises cyanogen bromide, formic acid, or thiotrifluoroacetic acid.

16. The method of claim 9, wherein the plurality of in silico proteolytic peptides comprise peptides having up to three missed enzymatic cleavage sites and ranging in molecular mass from 500 Da to 10,000 Da.

17. The method of claim 9, wherein the second database of protein sequences are derived from amino acid sequences encoded by one or more members of an EST library, a cDNA library, or a genomic library.

18. The method of claim 9, wherein providing the mass peak list further comprises contacting the sample with a first derivatizing agent, wherein the first derivatization agent comprises at least two isotopic forms, and specifically labels a selected amino acid or a functional moiety when the selected amino acid or functional moiety is present in a protein in the sample, thereby labeling the selected amino acid in one or more member proteins.

19. The method of claim 18, wherein contacting the sample with the first derivatizing agent is performed prior to generating the plurality of proteolytic peptides by action of the proteolytic agent.

20. The method of claim 18, wherein contacting the sample with the first derivatizing agent is performed after generating the plurality of proteolytic peptides by action of the proteolytic agent.

21. The method of claim 18, wherein the derivatizing agent comprises 2-methoxy-4,5-dihydro-1H-imidazole and the selected amino acid comprises lysine.

22. The method of claim 18, wherein providing the second list of theoretical masses comprises:
determining a number of occurrences of the selected amino acid or functional moiety in the in silico proteolytic peptides, thereby determining a number of derivatizing agents that would be attached to the in silico proteolytic peptides; and
calculating a theoretical molecular masses for the in silico proteolytic peptides having the determined number of attached derivatizing agents.

23. The method of claim 18, wherein each member of the second database of protein sequences comprises at least one selected amino acid.

24. The method of claim 9, wherein providing the mass peak list further comprises:
fractionating the sample to generate fractions comprising a plurality of peptides; and
ionizing member polypeptides in one or more of the fractions and obtaining masses using a mass spectrometer that provides a mass accuracy of 5 ppm or better.

25. The method of claim 24, wherein fractionating the sample comprises performing liquid chromatography, reverse phase chromatography, size exclusion chromatography, strong cation or anion exchange chromatography, weak cation or anion exchange chromatography, immobilized metal ion affinity chromatography (IMAC), capillary electrophoresis, gel electrophoresis, isoelectric focusing, or a combination thereof.

26. The method of claim 24, wherein ionizing the polypeptide comprises performing ESI.

27. The method of claim 24, wherein ionizing the polypeptide comprises performing LDI.

28. The method of claim 27, wherein the LDI comprises MALDI, IR-MALDI, UV-MALDI, liquid-MALDI, surface-enhanced LDI (SELDI), surface enhanced neat desorption (SEND), desorption/ionization of silicon (DIOS), laser desorption/laser ionization MS, or laser desorption/two step laser ionization MS.

29. The method of claim 24, wherein fractionating the sample further comprises depositing a plurality of fractions of an eluent onto a solid support suitable for laser desorption/ionization (LDI).

30. The method of claim 29, wherein the solid support comprises a surface modified for sample confinement.

31. The method of claim 24, wherein the mass spectrometer comprises a Fourier-transform ion cyclotron resonance mass spectrometer.

32. The method of claim 24, further comprising treating the sample to remove peptide modifications prior to the ionizing step.

33. The method of claim 24, wherein performing mass spectrometry further comprises providing one or more standards for comparison to the mass of the peak of interest, ionizing the one or more standards separately from the sample, thereby providing ionized standards, and mixing the ionized standards with an ionized sample in a gas phase.

34. The method of claim 1, wherein the sample comprises a proteome.

35. The method of claim 1, further comprising confirming an identification of a peak by tandem MS.

36. The method of claim 1, wherein calculating the first list of theoretical masses further comprises:
selecting a type of peptide modification; and
generating theoretical masses for the first set of in silico proteolytic peptides generated from the first database, wherein member proteins are assumed to contain one or more occurrences of the peptide modification, thereby identifying one or more peaks corresponding to modified member protein in the sample.

37. The method of claim 36, wherein the peptide modification comprises a post-translational modification as performed by a cell.

38. The method of claim 36, wherein the peptide modification comprises a chemical modification or an added chemical substituent.

39. The method of claim 36, wherein the peptide modification comprises a non-standard amino acid.

40. The method of claim 36, wherein the peptide modification comprises an amino acid substitution.

41. The method of claim 36, wherein the peptide modification comprises addition of one or more phosphate groups.

42. The method of claim 36, wherein the peptide modification comprises one or more myristoylate groups.

43. The method of claim 36, further comprising confirming an identification of a post-translationally modified protein by tandem MS of the member protein.

44. The method of claim 1, further comprising identifying member proteins corresponding to any remaining unidentified entries in the mass peak list by tandem MS.

45. A method of reducing a number of peaks to further be analyzed in a mass spectrum for a sample, the method comprising:
generating a first amino acid sequence database comprising an amino acid sequence of at least one protein present in the sample;
calculating a first list of theoretical masses for a first set of known in silico proteolytic peptides generated from the first database;
correlating a first theoretical mass with a position of an unidentified MS peak in a mass spectrum for the sample, thereby determining the presence in the sample of a first protein that comprises a peptide having a mass equal to the first theoretical mass; and
identifying one or more MS peaks that correspond to masses for the known in silico proteolytic peptides, thereby reducing the number of peaks to further be analyzed in the mass spectrum.

46. A method of identifying members of a plurality of proteins in a sample, the method comprising:
contacting a sample comprising a plurality of proteins with at least a first proteolytic agent that cleaves member proteins at defined cleavage sites to form proteolytic peptides;
contacting the sample with a first derivatizing agent comprising at least two isotopic forms, wherein the first derivatizing agent specifically labels a selected amino acid or functional moiety when the selected amino acid or functional moiety is present in a protein in the sample, thereby isotopically labeling one or more members of the plurality of proteins or proteolytic peptides;
fractionating the sample and depositing a plurality of fractions of an eluent onto a solid support suitable for LDI;
performing LDI-FT ICR mass spectrometry on the isotopically-labeled peptides in one or more of the fractions and determining masses of at least one pair of peaks of interest using a mass spectrometer that provides a mass accuracy of 5 ppm or better;
calculating a list of theoretical molecular masses for a plurality of in silico derivatized proteolytic peptides, wherein the member proteolytic peptides i) are derived from the amino acid sequences in a protein sequence database by predicted action of the proteolytic reagent upon members of the database; ii) encompass peptides having up to three missed proteolytic cleavage sites; iii) range in size between 1000 Da and 6000 Da; and iv) comprise one or more derivatized amino acids; and
correlating the list of theoretical molecular masses to the mass peak list of experimental mass peaks, wherein a match between an experimental mass peak of a sample proteolytic peptide and a theoretical molecular mass for an in silico proteolytic peptide is indicative of the presence in the sample of the protein from which the in silico proteolytic peptide is derived, thereby assigning MS peaks in the mass peak list and identifying the members of the plurality of proteins.

47. The method of claim 46, further comprising:
removing the assigned peaks from the mass peak list;
incorporating the identified members of the plurality of proteins into a database of identified proteins; and
repeating the calculation and correlating steps using in silico derivatized proteolytic peptides generated from the database of identified proteins, thereby assigning additional MS peaks in the mass peak list and identifying additional members of the plurality of proteins.

48. The method of claim 46, further comprising:
providing one or more additional databases of proteolytic peptide sequences, wherein the member proteolytic peptides i) are derived in silico by predicted action of one or more additional proteolytic reagents upon members sequences in the second database of protein sequences; ii) encompass peptide sequences having up to three missed enzymatic cleavage sites; iii) range in size between 1000 Da and 4000 Da; and iii) comprise one or more derivatized amino acids; and
repeating the generating and correlating step using the one or more additional databases, thereby identifying additional members of the plurality of proteins.

49. A method for identifying two or more members of a plurality of proteins in a sample, the method comprising:
a) providing a sample comprising a plurality of proteolytic polypeptides;
b) ionizing member polypeptides by LDI and obtaining a mass of at least a first polypeptide using a mass spectrometer that provides a mass accuracy of 5 ppm or better;
c) comparing the mass of the first polypeptide to members of a database of theoretical molecular masses for a plurality of in silico proteolytic peptides, wherein each member in silico peptide has a unique theoretical mass, and wherein a match between the mass obtained for the first polypeptide and the unique theoretical mass for an in silico proteolytic peptide indicates that a parent protein comprising the in silico polypeptide is present in the sample, thereby identifying a first protein in the sample; and
d) repeating the comparing step for one or more masses obtained for additional sample polypeptides, thereby identifying additional proteins in the sample.

50. The method of claim 49, wherein the plurality of proteins comprises a proteome or a sub-proteome.

51. The method of claim 50, wherein the proteome comprises a human proteome.

52. The method of claim 50, wherein the sub-proteome comprises a preparation of ribosomes, protein complexes, or organelles and comprises at least 50 proteins.

53. The method of claim 49, wherein the plurality of proteins comprises at least 1,000 proteins.

54. The method of claim 53, wherein the plurality of proteins comprises at least 25,000 proteins.

55. The method of claim 49, wherein the method identifies at least 50 percent of the proteins in the sample.

56. The method of claim 49, wherein providing the sample further comprises contacting the plurality of proteins with a first derivatizing agent, wherein the first derivatization agent comprises at least two isotopic forms and specifically labels a selected amino acid or functional moiety when the selected amino acid or functional moiety is present in a member protein.

57. The method of claim 56, wherein the derivatizing agent comprises 2-methoxy-4,5-dihydro-1H-imidazole.

58. The method of claim 59, wherein the derivatizing agent comprises a maleimide, a haloacetyl, an iodoacetamide, or a vinylpyridine.

59. The method of claim 56, wherein the selected amino acid comprises cysteine.

60. The method of claim 56, wherein the selected amino acid comprises lysine and wherein the derivatizing agent reacts with less than 10% of N-terminal amino groups.

61. The method of claim 56, wherein the selected amino acid comprises lysine and wherein the derivatizing agent reacts with less than 1% of N-terminal amino groups.

62. The method of claim 56, wherein the selected amino acid comprises an acidic amino acid, and wherein the derivatizing agent comprises acidic methanol.

63. The method of claim 56, wherein at least one isotopic form of the derivatizing agent is selected from the group consisting of deuterium, ¹³C, ¹⁴C, ¹⁵N, ¹⁸O, ³⁵Cl, ³⁷Cl, ⁷⁹Br and ⁸¹Br labeled agents.

64. The method of claim 56, wherein the theoretical molecular masses are obtained by:
i) determining a number of occurrences of the selected amino acid in the in silico proteolytic peptides, thereby determining a number of derivatizing agents that would be attached to the in silico proteolytic peptides; and
ii) calculating a theoretical molecular masses for the in silico proteolytic peptides having the determined number of attached derivatizing agents.

65. The method of claim 49, wherein providing the sample further comprises fractionating the sample.

66. The method of claim 65, wherein fractionating the sample comprises performing liquid chromatography, reverse phase chromatography, size exclusion chromatography, strong cation or anion exchange chromatography, weak cation or anion exchange chromatography, immobilized metal ion affinity chromatography (IMAC), capillary electrophoresis, gel electrophoresis, isoelectric focusing, or a combination thereof.

67. The method of claim 49, wherein fractionating the sample further comprises depositing a plurality of fractions of an eluent onto a solid support suitable for LDI.

68. The method of claim 67, wherein the solid support comprises a surface modified for sample confinement.

69. The method of claim 67, wherein the solid support comprises a hydrophobic/hydrophilic MALDI plate.

70. The method of claim 49, wherein ionizing member polypeptides by LDI comprises performing MALDI, IR-MALDI, UV-MALDI, liquid-MALDI, surface-enhanced LDI (SELDI), surface enhanced neat desorption (SEND), desorption/ionization of silicon (DIOS), laser desorption/laser ionization MS, or laser desorption/two step laser ionization MS.

71. The method of claim 49, wherein the mass spectrometer comprises a Fourier-transform ion cyclotron resonance mass spectrometer.

72. The method of claim 49, further comprising identifying predicted cleavage sites for a first proteolytic reagent in amino acid sequences of one or more proteins and determining amino acid sequences of one or more in silico proteolytic peptides that would be obtained by cleavage of the protein at one or more of the predicted cleavage sites.

73. The method of claim 49, further comprising:
e) calculating theoretical molecular masses for additional in silico peptides derived from the parent protein; and
f) repeating the comparing step for a mass obtained for a second peptide and disregarding mass spectral data for the second peptide if the mass spectral data for the second peptide matches that which would be obtained for one or more of the additional in silico peptides from the previously identified protein.

74. The method of claim 73, wherein the mass spectral data for the second peptide is disregarded if a mass obtained for the second peptide is within 5 ppm of the theoretical molecular mass of the additional in silico peptide derived from the previously identified protein; and if one or both of the following conditions apply:
an expression ratio determined for the second peptide corresponds to an expression ratio for the first peptide; and/or
a number of derivatized amino acids of the second peptide corresponds to a number of theoretical derivatized amino acids for the second in silico peptide.

75. The method of claim 49, wherein the in silico proteolytic peptides comprise peptides having up to three missed enzymatic cleavage sites and range in molecular mass from 500 Da to 10,000 Da.

76. The method of claim 75, wherein the in silico proteolytic peptides range in molecular mass from 1000 Da to 6000 Da.

77. The method of claim 49, wherein the in silico proteolytic peptides are derived from amino acid sequences encoded by one or more members of an EST library, a cDNA library, or a genomic library.

78. The method of claim 49, wherein the in silico proteolytic peptides are derived from amino acid sequences present in, or encoded by, one or more members of a human sequence library.

79. The method of claim 49, wherein the in silico proteolytic peptides are derived from amino acid sequences present in, or encoded by, one or more members of a yeast sequence library.

80. The method of claim 49, wherein the method further comprises:
identifying one or more fractions that contain a proteolytic peptide for which no unambiguous match was observed among the in silico proteolytic peptides; and
subjecting that fraction to further analysis to identify the proteolytic peptide that is present in the fraction.

81. The method of claim 80, wherein the further analysis comprises tandem MS.

82. The method of claim 49, further comprising:
e) contacting the sample with at least a first proteolytic reagent that cleaves proteins at defined cleavage sites to form sample proteolytic polypeptides.

83. The method of claim 82, wherein contacting the sample with the proteolytic agent is performed prior to contacting the sample with a first derivatizing agent.

84. The method of claim 82, wherein contacting the sample with the proteolytic agent is performed after contacting the sample with a first derivatizing agent.

85. The method of claim 82, wherein the proteolytic reagent comprises a proteolytic enzyme.

86. The method of claim 82, wherein the proteolytic enzyme is selected from the group consisting of trypsin, chymotrypsin, endoprotease ArgC, aspN, gluC, and lysC.

87. The method of claim 82, wherein the proteolytic reagent comprises cyanogen bromide, formic acid, or thiotrifluoroacetic acid.

88. The method of claim 82, further comprising treating the sample to remove post-translational modifications prior to subjecting the proteolytic peptides to mass spectrometry.

89. The method of claim 82, further comprising selecting a subset of proteolytic peptides comprise peptides having greater than 5 amino acids.

90. The method of claim 82, further comprising selecting a subset of proteolytic peptides comprise peptides having greater than 10 amino acids.

91. The method of claim 82, further comprising selecting a subset of proteolytic peptides comprise peptides having greater than 25 amino acids.

92. A method for identifying two or more proteins in a sample, the method comprising:
a) contacting a sample that comprises a plurality of proteins with at least a first proteolytic reagent that cleaves proteins at defined cleavage sites to form sample proteolytic peptides;
b) subjecting at least a first proteolytic peptide to mass spectrometry to determine a mass of the first proteolytic peptide;
c) comparing the mass determined for the first proteolytic peptide to theoretical molecular masses for a plurality of in silico proteolytic peptides that are derived from amino acid sequences for a plurality of proteins, wherein a match between the mass determined for the first proteolytic peptide and the theoretical molecular mass for an in silico proteolytic peptide is indicative of the presence in the sample of the protein from which the in silico proteolytic peptide is derived;
d) calculating theoretical molecular masses for additional in silico proteolytic peptides derived from the protein identified in the comparison of the mass determined for the first proteolytic peptide to the theoretical molecular masses; and
e) repeating the comparing step for a mass obtained for a second proteolytic peptide, and disregarding mass spectral data for the second proteolytic peptide if the mass spectral data is within 5 ppm of that which would be obtained for one or more of the additional in silico proteolytic peptides from the previously identified protein.

93. The method of claim 92, wherein the mass spectrometry is performed using a mass spectrometer that provides a mass accuracy of 5 ppm or better.

94. The method of claim 92, wherein the mass spectrometry comprises FT-ICR MS.

95. An integrated system for identifying a plurality of member proteins in a sample, the system comprising:
an ionization source and a mass spectrometer that provides a mass accuracy of 5 ppm or better;
an interface for receiving mass spectral data from the mass spectrometer, wherein the mass spectral data comprises mass peaks representing masses of a plurality of proteolytic peptides generated by treating the sample with at least a first proteolytic reagent;
a database of theoretical molecular masses of in silico-generated proteolytic peptides, wherein the peptides are derived by predicted action of the proteolytic reagent upon members of a database of protein sequences; and
a computer or computer-readable medium in communication with the interface and the database, the computer or computer-readable medium comprising instructions for determining a mass of a member proteolytic peptide from the mass spectral data and comparing the determined mass to members of the database of theoretical molecular masses, wherein a match between the mass determined for the proteolytic peptide and a theoretical molecular mass for an in silico proteolytic peptide is indicative of the presence in the sample of the protein from which the in silico proteolytic peptide is derived.

96. The system of claim 95, wherein the mass spectral data comprises mass peaks obtained from a sample that was contacted with at least a first amino acid derivatizing agent, and the system comprises instructions for adjusting the molecular mass determined for the in silico proteolytic peptide by adding to a calculated molecular mass the molecular mass of the derivatizing agent multiplied by the number of occurrences of a derivatized amino acid in the proteolytic peptide.

97. The system of claim 95, wherein the mass spectral data comprises mass peaks obtained from a sample that was contacted with at least a first amino acid derivatizing agent, and the system comprises instructions for adjusting the molecular mass determined for a proteolytic peptide by subtracting from the observed molecular mass for the proteolytic peptide the molecular mass of the derivatizing agent multiplied by the number of occurrences of a derivatized amino acid in the proteolytic peptide.

98. The system of claim 97, wherein the system comprises: a) instructions for generating a subset of in silico proteolytic peptides that comprise a selected amino acid to which the derivatizing agent can attach; b) instructions for calculating molecular masses for the subset of in silico proteolytic peptides having an attached derivatizing agent; and c) instructions for comparing the molecular masses for the derivatized in silico proteolytic peptides to the mass peaks for the sample proteolytic peptides.

99. The system of claim 95, wherein the mass spectrometer is an FT-ICR mass spectrometer.

100. The system of claim 95, wherein the plurality of proteins comprises a proteome or a sub-proteome.

101. The system of claim 100, wherein the proteome comprises a human or yeast proteome.

102. The system of claim 95, wherein the in silico proteolytic peptides encompass peptides having up to three missed enzymatic cleavage sites and range in size from 500 Da to 10,000 Da.

103. The system of claim 102, wherein the in silico proteolytic peptides range in molecular mass from 1000 Da to 6000 Da.

104. The system of claim 95, wherein the in silico proteolytic peptides each comprise at least 5 amino acids.

105. The system of claim 95, wherein the in silico proteolytic peptides each comprise at least 10 amino acids.

106. The system of claim 95, wherein the in silico proteolytic peptides each comprise at least 25 amino acids.

107. The system of claim 95, further comprising one or more additional databases of in silico proteolytic peptides, wherein the member in silico proteolytic peptides of the additional databases i) are derived in silico from the database of protein sequences by action of one or more additional proteolytic enzyme upon members of the database; ii) encompass peptide sequences having up to three missed enzymatic cleavage sites; and iii) range in size between 1000 Da and 4000 Da.

108. The system of claim 95, wherein the interface further comprises software for controlling generation and processing of the mass spectral data by the mass spectrometer.

109. The system of claim 95, further comprising a liquid chromatography system fluidically coupled to an automated sample collection system that comprises an eluent collection plate, wherein the mass spectrometer is configured to analyze ions generated from sample fractions present on the collection plate.

110. The system of claim 109, wherein the liquid chromatography system comprises a HPLC system.

111. The system of claim 109, wherein the eluent collection plate comprises a hydrophobic coating and one or more hydrophilic regions.

112. The system of claim 109, further comprising a sample source and a source of one or more proteolytic reagents, wherein the sample source and the source of proteolytic reagents are fluidically coupled to one another through a mixing region, and wherein the mixing region is fluidically coupled to the liquid chromatography system.

113. The system of claim 112, wherein one or more of the sample source, the source of proteolytic reagents, and the mixing region comprise microtiter plate wells.

114. The system of claim 112, wherein one or more of the sample source, the source of proteolytic reagents, the mixing region, and the liquid chromatography system are incorporated into a microfluidic device.

115. The system of claim 95, wherein the system comprises instructions for:
calculating theoretical molecular masses for additional in silico proteolytic peptides derived from the protein identified in the comparison of the mass obtained for the first proteolytic peptide to the theoretical molecular masses; and
disregarding mass spectral data for a second proteolytic peptide if a determined mass for the second proteolytic peptide matches a theoretical molecular mass for an additional in silico proteolytic peptides derived from the previously identified protein.

116. The system of claim 95, wherein the computer or computer readable medium sequentially compares two or more sample masses to the theoretical molecular masses for the in silico proteolytic peptides.

117. The system of claim 95, wherein the computer or computer readable medium simultaneously compares two or more sample masses to the theoretical molecular masses for the in silico proteolytic peptides.
